SEQUENCE LISTING (18 sequences)

SEQ ID NO: 1 - DNA, 21 bp, Artificial Sequence
  Synthesised forward primer of mUCP1
aggcttccagtaccattaggt

SEQ ID NO: 2 - DNA, 22 bp, Artificial Sequence
  Synthesised reverse primer of mUCP1
ctgagtgaggcaaagctgattt

SEQ ID NO: 3 - DNA, 19 bp, Artificial Sequence
  Synthesised forward primer of mpgc1a
ccctgccattgttaagacc

SEQ ID NO: 4 - DNA, 19 bp, Artificial Sequence
  Synthesised reverse primer of mpgc1a
tgctgctgttcctgttttc

SEQ ID NO: 5 - DNA, 20 bp, Artificial Sequence
  Synthesised forward primer of mprdm16
tcccaccagacttcgagcta

SEQ ID NO: 6 - DNA, 20 bp, Artificial Sequence
  Synthesised reverse primer of mprdm16
atctcccatccaaagtcggc

SEQ ID NO: 7 - DNA, 21 bp, Artificial Sequence
  Synthesised forward primer of mBMP7
gagggctggttggtgtttgat

SEQ ID NO: 8 - DNA, 21 bp, Artificial Sequence
  Synthesised reverse primer of mBMP7
gttgcttgttctggggtccat

SEQ ID NO: 9 - DNA, 20 bp, Artificial Sequence
  Synthesised forward primer of m beta-actin
gaagtccctcaccctcccaa

SEQ ID NO: 10 - DNA, 16 bp, Artificial Sequence
  Synthesised reverse primer of m beta-actin
ggcatggacgcgacca

SEQ ID NO: 11 - DNA, 20 bp, Artificial Sequence
  Synthesised forward primer of m PPAR g
tcgctgatgcactgcctatg

SEQ ID NO: 12 - DNA, 21 bp, Artificial Sequence
  Synthesised reverse primer of m PPAR g
gagaggtccacagagctgatt

SEQ ID NO: 13 - DNA, 21 bp, Artificial Sequence
  Synthesised forward primer of m c-EBP a
caagaacagcaacgagtaccg

SEQ ID NO: 14 - DNA, 21 bp, Artificial Sequence
  Synthesised reverse primer of m c-EBP a
gtcactggtcaactccagcac

SEQ ID NO: 15 - DNA, 22 bp, Artificial Sequence
  Synthesised forward primer of m FAS
ctgagatcccagcacttcttga

SEQ ID NO: 16 - DNA, 19 bp, Artificial Sequence
  Synthesised reverse primer of m FAS
gcctccgaagccaaatgag

SEQ ID NO: 17 - DNA, 18 bp, Artificial Sequence
  Synthesised forward primer of m AP2
catggccaagcccaacat

SEQ ID NO: 18 - DNA, 20 bp, Artificial Sequence
  Synthesised reverse primer of m AP2
cgcccagtttgaaggaaatc

(19) United States
(12) Patent Application Publication
(10) Pub. No.: US 2020/0030388 A1
(43) Pub. Date: Jan. 30, 2020

(54) COMPOSITIONS FOR MODIFYING GUT MICROBIOTA

(71) Applicants: Muhammed Majeed, Banaglore (IN); Kalyanam Nagabhushanam, East Windsor, NJ (US); Lakshmi Mundkur, Bangalore (IN)

(72) Inventors: Muhammed Majeed, Banaglore (IN); Kalyanam Nagabhushanam, East Windsor, NJ (US); Lakshmi Mundkur, Bangalore (IN)

(21) Appl. No.: 16/587,310

(22) Filed: Sep. 30, 2019

Related U.S. Application Data
(63) Continuation-in-part of application No. 16/007,212, filed on Jun. 13, 2018.
(60) Provisional application No. 62/519,949, filed on Jun. 15, 2017.
(60) Provisional application No. 62/523,611, filed on Jun. 22, 2017.

(51) Int. Cl. A61K 35/741; A61P 1/00

(52) U.S. Cl. CPC A61K 35/741; A61K 45/06; A61P 1/00

(57) ABSTRACT

The present invention pertains to a composition comprising garcinol individually or in combination with forskolin for modifying the gut microbial diversity. More specifically, the invention relates to the use of composition comprising garcinol and forskolin for decreasing the Firmicutes/Bacteroidetes ratio and increasing the count of beneficial microbe, Akkermansia muciniphila.

### CROSS-REFERENCE TO RELATED PATENT APPLICATION

The present invention is a continuation in part application of US complete application number 16007212 deriving priority from U.S. provisional patent applications No. 62/519,949 filed on 15 Jun. 2017 and No. 62/523,611 filed on 22 Jun. 2017, incorporated herein in its entirety.

### BACKGROUND OF THE INVENTION

### Field of the Invention

The invention in general relates to compositions for maintenance or management of gut health. Specifically the invention relates to compositions comprising garcinol individually or in combination with forskolin for modifying the viable counts of gut microbiota population. More specifically the invention targets increasing colony count of Akkermansia muciniphila in the gut using composition comprising garcinol individually or in combination with Forskolin.

### Description of Prior Art

Obesity is considered to be the leading health risk for the development of various disorders like hypertension, type 2 diabetes, heart disease, stroke, osteoarthritis, and mental illness. Globally, more than 1 in 10 individuals are obese and about 36% of American adults are obese (https://www.medicalnewstoday.com/articles/319902.php, accessed on 10 May 2018). Obesity results due to imbalance between the energy content of food eaten and energy expended by the body to maintain life and to perform physical work. Such an energy balance framework is a potentially powerful tool for investigating the regulation of body weight.

Recently, it was observed that the gut microbiota is altered in conditions like obesity and type II diabetes. The gut microbiota can affect the absorption of nutrients and energy distribution in the host, these gut microbiota are important in the pathogenesis of disease such as obesity and related metabolic syndromes (Zhao, L. (2013) The gut microbiota and obesity: from correlation to causality, Nature Reviews Microbiology, 11(9), 639). Administration of probiotics to obese individuals resulted in an effective weight loss. One particular gut microbe, Akkermansia muciniphila was inversely associated with obesity, diabetes, cardiometabolic diseases and low-grade inflammation (Cani et al., Next-Generation Beneficial Microbes: The Case of Akkermansia muciniphila, Front. Microbiol., 22 Sep. 2017, https://doi.org/10.3389/finicb.2017.01765). Evidence show that the relative abundance of A. muciniphila increased more than 100-fold following the ingestion of prebiotics (Everard et al., 2014 Microbiome of prebiotic-treated mice reveals novel targets involved in host response during obesity. ISME J. 8, 2116-2130. doi: 10.1038/ismej2014.45). Studies also indicated that the number of A. muciniphila was found to be lower in obese and type 2 diabetic mice and increased with antidiabetic treatments (Cani et al., Next-Generation Beneficial Microbes: The Case of Akkermansia muciniphila, Front. Microbiol., 22 Sep. 2017, https://doi.org/10.3389/fmicb.2017.01765). Another study observed that A. muciniphila treatment reversed high-fat diet-induced metabolic disorders, including fat-mass gain, metabolic endotoxemia, adipose tissue inflammation, and insulin resistance. (Amandine Everard, Clara Belzer, Lucie Geurts, Janneke P. Ouwerkerk, Cbline Druart, Laure B. Bindels, Yves Guiot, Muriel Derrien, Giulio G. Muccioli, Nathalie M. Delzenne, Willem M. de Vos and Patrice D. Cani, Cross-talk between Akkermansia muciniphila and intestinal epithelium controls diet-induced obesity, PNAS May 28, 2013. 110 (22) 9066-9071). Hence, increasing the viable counts of Akkermansia muciniphila can be an effective therapy for the management of obesity, diabetes and other metabolic disorders. The probiotic and beneficial effects of Akkermansia muciniphila are well described in the following prior art documents.
1. Cani et al., Next-Generation Beneficial Microbes: The Case of Akkermansia muciniphila, Front. Microbiol., 22 Sep. 2017, https://doi.org/10.3389/fmnicb.2017.01765
2. Gómez-Gallego et al., Akkermansia muciniphila: a novel functional microbe with probiotic properties, Beneficial Microbes, 2016; 7(4): 571-584

Natural molecules are currently evaluated extensively for the management of obesity and related disorders. Extracts of Garcinia cambogia, have been reported to have a weight loss potential. U.S. Pat. No. 7,063,861, discloses a weight loss composition containing garcinol and hydroxycitric acid (HCA) and optionally with anthocyanins. U.S. Pat. No. 8,329,743 also discloses a weight loss formulation containing garcinol, pterostilbene and anthocyanin. U.S. Pat. No. 7,063,861 indicates that garcinol and HCA combination increases the bioavailability of HCA bringing about an anti-obesity effect. Hence, the anti-obesity effect of garcinol per se is not reported and also cannot be anticipated from the prior art documents. Moreover, Heo et al., (Gut microbiota Modulated by Probiotics and Garcinia cambogia Extract Correlate with Weight Gain and Adipocyte Sizes in High Fat-Fed Mice Sci Rep. 2016; 6:33566), reports the modulation of gut microbiota and increase in A. muciniphila by Garcinia cambogia extract without specific reference to garcinol. The present invention solves the above problem by disclosing the ability of modulating the gut microbe by garcinol individually or in combination with Forskolin. More specifically the change in gut microbiota is observed is by modulating the count of Akkermansia muciniphila in the gut.

Forskolin, a diterpene, is the main ingredient extracted from the roots of Coleus forskohlii (Kamohara, S. (2016). An evidence-based review: Anti-obesity effects of Coleus forskohlii. Personalized Medicine Universe, 5, 16-20.). Previous studies have demonstrated that forskolin is an agonist of adenylate cyclase, which can directly activate adenylate cyclase and promote the decomposition of TG stored in adipocytes (Litosch, I., Hudson, T., Mills, I., Li, S., & Fain, J. (1982). Forskolin as an activator of cyclic AMP accumulation and lipolysis in rat adipocytes. Molecular pharmacology, 22(1), 109-115.). The effect of forskolin along with other ingredients has been reported to promote lean body mass in mammal and targeting adipose tissues to promote weight loss in application number EP977564A titled “Forskolin for Promoting Lean Body Mass” by Majeed et. al

In yet another prior art application number CA2649477C, titled “Composition and method for promoting internal health and external appearance” wherein forskolin is used in a composition with other botanical extracts promotes DNA repair, reduces lean body mass and reduces body fat levels.

Despite the known anti-obesity effects of forskolin from Coleus forskolin and garcinol from Garcinia indica, the combinational effect of these two compounds has not been reported. Specifically, reports on the ability of a composition comprising garcinol and forskolin to modify the composition of gut microbiota is not available.

The principle objective of the invention is to disclose a composition containing garcinol alone or in combination with forskolin to bring change in the gut microbiota thus maintaining gut health.

It is another objective of invention to disclose the ability of garcinol individually or in combination with forskolin to modify the gut microbial diversty and decreasing the Firmicutes/Bacteroidetes ratio and increasing the viable counts of probiotic bacteria Akkermansia muciniphila.

The present invention fulfils the above such objectives and provides further related advantages.

### SUMMARY OF THE INVENTION

The present invention pertains to a composition comprising garcinol individually or in combination with forskolin for modifying the gut microbial diversity. More specifically, the invention relates to the use of composition comprising garcinol and forskolin for decreasing the Firmicutes/Bacteroidetes ratio

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1A is the oil-O-red staining of adipocytes indicating a dose dependent reduction in lipid accumulation in adipocytes by garcinol.

FIG. 1B is the graphical representation of the percentage inhibition of adipogenesis by garcinol.

FIG. 2 is a graphical representation showing the decrease in expression of genes related to adipogenesis in garcinol treated groups.

FIG. 3 is a graphical representation showing the increase in expression of genes related to brown fat conversion and fat utilization in garcinol treated groups.

FIG. 4A is a graphical representation showing the change in weight of animals administered with different concentrations of garcinol over a period of 4 months.

FIG. 4B is graphical representation showing the final weight of animals administered with different concentrations of garcinol for a period of 120 days.

FIG. 5 is a representative image showing the different fat pad regions in the mice body.

FIG. 6 represents the change in the weight of peritoneal, mesenteric and perigonadal fat tissues treated with different concentrations of garcinol.

FIG. 7 is a graphical representation showing the percentage reduction in visceral fat in animals administered with different concentrations of garcinol in a dose dependent manner.

FIGS. 8A and 8B are graphical representations showing the decrease in expression of genes related to adipogenesis in adipose tissues of animals administered with different concentrations of garcinol.

FIG. 9 is a graphical representation showing the increase in expression of genes related to brown fat conversion and fat utilisation in adipose tissues of animals administered with different concentrations of garcinol.

FIG. 10A is a graphical representation showing the levels of total cholesterol and triglycerides in serum of animals administered with different concentrations of garcinol.

FIG. 10B is a graphical representation showing the levels of LDL and VLDL in serum of animals administered with different concentrations of garcinol.

FIG. 10C is a graphical representation showing the levels of HDL in serum of animals administered with different concentrations of garcinol.

FIG. 11 shows the experimental design for anti obesity study with Garcinol in HFD-induced Obesity Mice.

FIG. 12 is a representative image showing the effect of Garcinol on HFD-induced Obesity in C57BL/6 mice. Image A is the representative photographs of each group of mice at the end of week 13. Image B shows the Photographs of perigonadal adipose tissues and image C shows photographs of the liver.

FIG. 13 is a graphical representation of body weight of animal administered with various concentrations of garcinol. Body weight was monitored weekly and the average body weight of each group was expressed as the means±SE, p<0.05; a, b, c, and d significantly differed between each group.

FIG. 14A shows the photographs of perigonadal, retroperitoneal, and mesenteric adipose tissues of animals administered with garcinol.

FIG. 14B is the graphical representation of adipose tissue weights of animals administered with garcinol.

FIG. 15A shows the representative images of each study group for the pathological assessment by H&E staining in perigonadal adipose tissue.

FIG. 15B is graphical representation showing the percentage frequency of adipocyte size on animals treated with garcinol. Adipocyte size was quantified under the microscope from representative sections.

FIGS. 16A and 16B show the change in the taxonomic composition of colonic bacterial communities in animals administered with garcinol. FIG. 16A shows the change in the phylum and FIG. 16B represents the genus relative abundance of fecal microbiota.

FIGS. 17A and 17B represents Principal Coordinate Analysis (PCoA) plots showing the normalized relative abundance of all samples (A) Phylum. (B) Genus

FIG. 17C represents the Heatmap showing the abundance of 50 operational taxonomic units (OTUs) significantly altered by garcinol in HFD-fed mice.

FIG. 18A shows the effects of garcinol on protein expression of adipocyte specific factors and AMPK signaling in HFD-fed C57BL/6 Mice Perigonadal Adipose Tissue. The protein levels of p-AMPK (Thr172), AMPK, Pref-1, SREBP-1 and PPARγ were determined by Western blot analysis with specific antibodies. β-actin was used as a loading control.

FIG. 18B is the graphical representation of the level of protein expression of adipocyte specific factors and AMPK signaling in HFD-fed C57BL/6 Mice Perigonadal Adipose Tissue. The values indicate the relative density of the protein band normalized to β-actin.*p<0.05;**p<0.005; compared with the HFD treatment only.

FIG. 19A is a graphical representation of body weight of animal administered with various concentrations of garcinol and garcinol blend.

FIG. 19B is the representative photographs of each group of mice at the end of the study period.

FIG. 20A is a graphical representation of perigonadal fat weights of animals administered with different concentrations of garcinol and garcinol blend.

FIG. 20B is a graphical representation of retroperitoneal fat weights of animals administered with different concentrations of garcinol and garcinol blend.

FIG. 20C is a graphical representation of mesenteric fat weights of animals administered with different concentrations of garcinol and garcinol blend.

FIG. 21A is a graphical representation showing the effect of garcinol, forskolin and composition comprising garcinol and forskolin (LMIX and HMIX) on the perigonadal fat weight of HFD-fed C57BL/6 mice.

FIG. 21B is a graphical representation showing the effect of garcinol, forskolin and composition comprising garcinol and forskolin (LMIX and HMIX) on the mesentric fat weight of HFD-fed C57BL/6 mice.

FIG. 21C is a graphical representation showing the effect of garcinol, forskolin and composition comprising garcinol and forskolin (LMIX and HMIX) on the retroperitonial fat weight of HFD-fed C57BL/6 mice.

FIG. 21D is a graphical representation showing the effect of garcinol, forskolin and composition comprising garcinol and forskolin (LMIX and HMIX) on the body fat ratio of HFD-fed C57BL/6 mice.

FIG. 21E is a histochemical image showing the effect of garcinol, forskolin and composition comprising garcinol and forskolin (LMIX and HMIX) on the size of adipocytes, as evaluated by H&E stain in the liver of HFD-fed C57BL/6 mice.

FIG. 21F is a histochemical image showing the effect of garcinol, forskolin and composition comprising garcinol and forskolin (LMIX and HMIX) on lipid accumulation in the liver of HFD-fed C57BL/6 mice.

FIG. 22A is a western blot image showing the expression of proteins PPARγ and C/EBPα in mice treated with garcinol, forskolin and composition comprising garcinol and forskolin (LMIX and HMIX).

FIG. 22B is a graphical representation showing the effect of garcinol, forskolin and composition comprising garcinol and forskolin (LMIX and HMIX) on the expression of PPARγ and C/EBPα in HFD-fed C57BL/6 mice.

FIG. 22C is a western blot image showing the expression of proteins p-HSL, HSL and p-PKA substrate in mice treated with garcinol, forskolin and composition comprising garcinol and forskolin (LMIX and HMIX).

FIG. 22D is a graphical representation showing the effect of garcinol, forskolin and composition comprising garcinol and forskolin (LMIX and HMIX) on the expression of p-HSL, HSL and p-PKA substrate in HFD-fed C57BL/6 mice.

FIG. 22E is a western blot image showing the expression of CPT-1A and PPARα in mice treated with garcinol, forskolin and composition comprising garcinol and forskolin (LMIX and HMIX).

FIG. 22F is a graphical representation showing the effect of garcinol, forskolin and composition comprising garcinol and forskolin (LMIX and HMIX) on the expression of CPT-1A and PPARα in HFD-fed C57BL/6 mice.

FIG. 23A is a venn diagram showing the effect of garcinol, forskolin and composition comprising garcinol and forskolin (LMIX and HMIX) on the operational taxonomic unit (OTU) numbers of the gut microbiota in HFD-fed C57BL/6 mice.

FIG. 23B is a graphical representation showing the effect of garcinol, forskolin and composition comprising garcinol and forskolin (LMIX and HMIX) on the relative abundance of gut microbiota in HFD-fed C57BL/6 mice.

FIG. 23C is a graphical representation showing the effect of garcinol, forskolin and composition comprising garcinol and forskolin (LMIX and HMIX) on the Firmicutes/Bacteroidetes ratio in HFD-fed C57BL/6 mice.

FIG. 23D is a graphical representation showing top 35 bacterial taxa at phylum level in gut of mice treated with garcinol, forskolin and composition comprising garcinol and forskolin (LMIX and HMIX).

FIG. 23E is a graphical representation showing top 35 bacterial taxa at genus level in gut microbiota of mice treated with garcinol, forskolin and composition comprising garcinol and forskolin (LMIX and HMIX).

FIG. 24A is a graphical representation showing the difference of relative abundance at genus level in gut microbiota of mice treated with high fat diet.

FIG. 24B is a graphical representation showing the difference of relative abundance at genus level in gut microbiota of mice treated with forskolin along with high fat diet.

FIG. 24C is a graphical representation showing the difference of relative abundance at genus level in gut microbiota of mice treated with garcinol and forskolin (LMIX).

FIG. 24D is a graphical representation showing the difference of relative abundance at genus level in gut microbiota of mice treated with garcinol and forskolin (HMIX).

FIG. 24E is a graphical representation showing the difference of relative abundance at species level in gut microbiota of mice treated with high fat diet.

FIG. 24F is a graphical representation showing the difference of relative abundance at species level in gut microbiota of mice treated with garcinol along with high fat diet.

FIG. 24G is a graphical representation showing the difference of relative abundance at species level in gut microbiota of mice treated with forskolin along with high fat diet.

FIG. 24H is a graphical representation showing the difference of relative abundance at species level in gut microbiota of mice treated with garcinol and forskolin (LMIX) along with high fat diet.

FIG. 24I is a graphical representation showing the difference of relative abundance at species level in gut microbiota of mice treated with garcinol and forskolin (HMIX) along with high fat diet.

### DESCRIPTION OF THE MOST PREFERRED EMBODIMENTS

In the most preferred embodiment, the present invention discloses a method for therapeutic management of obesity in mammals, said method comprising steps of administering effective concentration of a composition containing garcinol to said mammals to bring about a) inhibition of adipogenesis b) decrease in body weight and visceral fat in said mammals. In a related embodiment, inhibition of adipogenesis in brought about by down regulation of genes selected from the group consisting of, but not limited to, peroxisome proliferator-activated receptor gamma (PPARγ), CCAAT/enhancer binding protein alpha (cEBPα), first apoptotic signal (FAS), adipocyte protein 2 (AP2), resistin and leptin. In a related embodiment, inhibition of adipogenesis is brought about by up regulation of genes selected from the group consisting of, but not limited to, phospho-adenosine monophosphate-activated protein kinase (p-AMPK), AMP-activated protein kinase (AMPK) and Preadipocyte factor I (PREF-1). In another related embodiment, the visceral fat is selected from the group consisting of mesenteric fat, peritoneal fat and perigonadal fat. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method of achieving energy balance in mammalian adipose cellular systems, said method comprising step of administering composition containing garcinol in effective amounts targeted towards mammalian pre-adipocytes and/or adipocytes to achieve effects of (a) increased inhibition of adipogenesis and (b) increased expression of factors that function individually or in combination to specifically recruit brown adipocytes or brown like (beige or brite) adipocytes, c) induce brown like phenotype (beige or brite adipocytes) in white adipocyte depots, to bring about the effect fat utilization and energy balance in said mammals. In related embodiments, the factors include the transmembrane protein mitochondrial uncoupling protein (UCP-1), the transcription coregulators PR domain containing protein 16 (PRDM16) and Peroxisome proliferator-activated receptor gamma coactivator 1-alpha (PGC-1α) which regulate the genes involved in energy metabolism and bone morphogenic protein 7 (BMP7), secretory protein controlling energy expenditure. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the present invention discloses a method of modifying the gut microbiota composition in mammals, said method comprising step of administering effective amounts of a composition containing garcinol to said mammals to bring about change in the gut microbiota composition. In a related embodiment, the gut microbiota is selected from the Phylum Deferribacteres, Proteobacteria, Bacteroidetes, Verrucomicrobia and Firmicutes, Actinobacteria, Fusobacteria, Denerribacteres, Proteobacteria, Bacteroidetes, and Verrucomicrobia. In another related embodiment, the gut microbiota is selected from the genus Lactobacillus, Butyrivibrio, Clostridium, Anaerobranca, Dysgonomonas, Johnsonella, Ruminococctus, Bacteroides, Oscillospira, Parabacterroides, Akkermanisa, and Blautia, Neisseria. More specifically, the gut microbiota is selected from the group consisting of Parabacteroides goldsteinii, Bacteroides caccae, Johnsonella ignava, Blautia wexlerae, Dysgonomonas wimpennyi, Blautia hansenni, Anaerobranca zavarzinni, Oscillospira eae, Mucispirillus schaedleri, Blautia coccoides, Anaerotruncus colihominis, Butyrivibro proteoclasticus, Akkermansia muciniphila, Lachnospora pectinoschiza, Pedobacter kwangvangensis, Alkaliphilus crotonatoxidans, Lactobacillus salivarius, Anaerivibria lipolyticus, Rhodothermus clarus, Bacteroides stercorirosoris, Ruminocococcus flavefaciens, Bacteroides xylanisolvens, Ruminococcus gnavus, Clostridium termitidis, Clostridium alkalicellulosi, Emticicia oligoraphica, Pseudobutyrivibro xylanivorans, Aclinomyces naturae, Peptoniphilus coxii, and Dolichospermum curvum, Synergisletes, Gemmatimonadetes, Cyanobacteria, Acidobacteria, Fibrobacteres, Nitrrospirae, Thermomicrobia, Chlorobi, Chloroflexi, Actinobacteria, Fusobacteria, Elusimicribia, Tenericutes, Firmicutes, Saccharibacteria, Verrucomicrobia, Sphrochaetes, Absconditabacteria, Gracillibacteria, Bacteriodeles, Ruminoclostridium, Roseburia, Desulfovibrio, Anaerotruncus, Lautropia, Rumonococcaceae, Haeemphilus, Capnocytophaga, rothia, Prevotella, Porphyromonas, Lachnoanaermbaculum, Veillonella, Alloprevotella, Prevotella. In a related embodiment, modification of gut microbiota is effective in therapeutic management of diseases selected from the group consisting of obesity, cardiovascular complications, inflammatory bowel disease, Crohn's disease, Celiac disease, metabolic syndrome, liver diseases and neurological disorders. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the present invention discloses a method of modifying the gut microbiota composition in mammals, said method comprising step of administering effective amounts of a composition comprising garcinol individually or in combination with forskolin to said mammals to bring about change in the gut microbial diversity. In a related embodiment, the gut microbiota is selected from the Phylum Deferribacteres, Proteobacteria, Bacteroidetes, Verrucomicrobia, Actinobacteria, Fusobacteria, and Firmicutes. In a related embodiment, the composition comprising garcinol individually or in combination with forskolin decreases the Firmicutes/Bacteroidetes ratio. In another related embodiment, the gut microbiota is selected from the genus Lactobacillus, Butyrivibrio, Clostridium, Anaerobranca, Dysgonomonas, Johnsonella, Ruminococcus, Bacteroides, Oscillospira, Parabacterroides, Akkermanisa, Blautia, Neisseria, Synergistetes, Gemmatimonadetes, Cyanobacteria, Acidobacteria, Fibrobacteres, Nitrrospirae, Thermomicrobia, Chlorobi, Chloroflexi, Actinobacteria, Fusobacteria, Elusimicribia, Tenericutes, Firmicutes, Saccharibacteria, Verrucomicrobia, Sphrochaetes, Absconditabacteria, Gracillibacteria, Bacteriodetes, Ruminoclostridium, Roseburia, Desulfovibrio, Anaerotruncus, Lautropia, Rumonococcaceae, Haeemphilus, Capnocytophaga, rothia, Prevotella, Porphyromonas, Lachnoanaerobaculum, Veillonella and Alloprevotella. More specifically, the gut microbiota species is selected from the group comprising of Parabacteroides goldsteinii, Bacteroides caccae, Johnsonella ignava, Blautia wexlerae, Dysgonomonas wimpennyi, Blautia hansenni, Anaerobranca zavarzinni, Oscillospira eae, Mucispirillus schaedleri, Blautia coccoides, Anaerotruncus colihominis, Butyrivibro proteoclasticus, Akkermansia muciniphila, Lachnospora pectinoschiza, Pedobacter kwangyangensis, Alkaliphilus crotonatoxidans, Lactobacillus salivarius, Anaerivibria lipolyticus, Rhodothermus clarus, Bacteroides stercorirosoris, Ruminocococcus flavefaciens, Bacteroides xylanisolvens, Ruminococcuc gnavus, Clostridium termitidis, Clostridium alkalicellulosi, Emticicia oligoraphica, Pseudobutyrivibro xylanivorans, Actinomyces naturae, Peptoniphilus coxii, and Dolichospermum curvum. In a related embodiment, modification of gut microbial diversity is effective in therapeutic management of diseases selected from the group comprising of obesity, cardiovascular complications, inflammatory bowel disease, Crohn's disease, Celiac disease, metabolic syndrome, liver diseases and neurological disorders. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method for increasing the viable counts of Akkermansia muciniphila in the gut of mammals, said method comprising steps of administering effective amounts of a composition containing garcinol to mammals to bring about an increase in the colonies of said bacteria. In a related embodiment, the increase in the colony counts of Akkermansia muciniphila reduces body weight through the AMPK signaling pathway by causing endocannabinoid release. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method for increasing the viable counts of Akkermansia muciniphila in the gut of mammals, said method comprising steps of administering effective amounts of a composition comprising garcinol individually or in combination with forskolin to mammals to bring about an increase in the colonies of said bacteria. In a related embodiment, the increase in the colony counts of Akkermansia muciniphila reduces body weight through the AMPK signaling pathway by causing endocannabinoid release.

In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a method of therapeutic management of hyperlipidemia in mammals, said method comprising step of administering an effective concentration of a composition containing garcinol to bring about the effects of (i) reducing the amount of total blood cholesterol levels; (ii) reducing the concentrations of low density lipoproteins (LDL) and very low density lipoproteins (VLDL); (iii) increasing the concentrations of high density lipoproteins (HDL) and (iv) reducing concentrations of serum triglycerides, in the blood of said mammals. In a related embodiment, the medical cause of hyperlipidemia is obesity. In a related embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a composition containing garcinol for use as a prebiotic agent.

In another preferred embodiment, the invention discloses a composition containing garcinol individually or in combination with forskolin for use as a prebiotic agent. The terms “microbiome” and “microbiota” are used alternatively in the specification describes the entire population of micro organisms with in the gut of a mammal. Microbiome comprises all genetic material that is present in the microbiota.

Specific illustrative examples enunciating the most preferred embodiments are included herein below

### Example 1: Anti-Obesity Effects of Garcinol—Study Done at Sami Labs Limited, Bangalore, India and Srimad Andavan Arts & Science College, Tiruchirapalli, India

Adipogenesis Inhibition and Brown Fat Specific Gene Expression by Garcinol in Cultured 3T3L1 and Animal Tissues

Methodology

Preparation of Stock Solutions

Garcinol (20%) stock of 10 mg/ml was prepared in DMSO and filtered through 0.2 micron syringe filter. Stock was diluted 1000 times in DMEM to get 10 μg/ml final concentration and serially diluted. Insulin (Hi Media) was bought as a solution at a concentration of 20 mg/ml. This was diluted to 1 μg/ml in DMEM. IBMX-(Sigma)—Stock of 5 mM was prepared in DMEM, and diluted 10 times to be used at a final concentration of 0.5 mM. Dexamethasone (Sigma)—A stock of 10 μM was prepared in DMEM and diluted 40 times to get a final concentration of 0.25 μM

Cell Culture

Mouse 3T3-L1 pre-adipocytes were cultured in DMEM containing 25 mM glucose with 10% heat-inactivated fetal calf serum with antibiotics at 37° C. and 5% CO2. When the cells were 70-80% confluent, they were trypsinized, washed and seeded in 6 well plates at a density of 2×10⁶ cells per well. Cells were induced to differentiate 2 d after reaching confluence (day 0), by supplementing DMEM media containing 10% Fetal Bovine Serum (FBS) along with 1 μg/mL insulin, 0.25 μM dexamethasone, 0.5 mM 1-methyl-3-isobutyl-xanthine (IBMX) and different concentrations of Garcinol (20%). From day 3 until day 7, cells were maintained in progression media supplemented with 1 μg/mL insulin and different concentrations of Garcinol (20%). Untreated cells and undifferentiated cells grown in FCS media were taken as Adipogenesis positive and negative controls for the experiment. Quantification for amount of triglycerides accumulated in adipocytes was done by Oil red O staining.

RNA Extraction

Cells were harvested after second progression on day 7 and total RNA was extracted using the Trizol method. Extracted RNA was treated with DNAse I to remove any contaminating DNA and again extracted using phenol: chloroform: isoamyl alcohol extraction (24:25:1). Quality of RNA was determined by checking the absorbance at 260/280 nm using a Nanodrop (Thermo)

Gene Expression Studies in Mouse Fat Pad

The frozen fat pads from treated and untreated animals were collected in RNA later and frozen. Approximately 100 mg of the tissue was homogenized in ice and extracted with 1 ml Trizol as described earlier.

Quantitative Real Time PCR

2 μg of total RNA was taken for cDNA synthesis using SuperScript III First-Strand Synthesis System (Life Technologies). Quantitative RT-PCR analysis was performed to determine the expression of brown fat specific genes in Roche Light cycler 96 using SYBR Green master mix (Thermo Scientific). β actin was used as a house keeping gene The relative RNA abundance of BAT genes was normalized to the housekeeping β actin gene and expressed as delta delta CT (equivalent to fold change transformed by Log₂).

Primer sequence: The primers used for the determining the expression of brown fat specific genes and genes related to adipogenesis is given in table 1

TABLE 1
Primers used for analyzing the expression of BAT
and adipogenesis specific genes

| | |
|---|---|
| Name | Primer sequence |

BAT specific Genes

| | |
|---|---|
| m Ucp1 F | AGGCTTCCAGTACCATTAGGT |
| | (SEQ ID #1) |
| m Ucp1 R | CTGAGTGAGGCAAAGCTGATTT |
| | (SEQ ID #2) |
| mpgc1αaF | CCC TGC CAT TGT TAA GAC C |
| | (SEQ ID #3) |
| mpgc1αaR | TGC TGC TGT TCC TGT TTT C |
| | (SEQ ID #4) |
| mprdm16 F | TCCCACCAGACTTCGAGCTA |
| | (SEQ ID #5) |
| mprdm16 R | ATCTCCCATCCAAAGTCGGC |
| | (SEQ ID #6) |
| mBMP7 F | GAGGGCTGGTTGGTGTTTGAT |
| | (SEQ ID #7) |
| mBMP7 R | GTTGCTTGTTCTGGGGTCCAT |
| | (SEQ ID #8) |
| m βactin F | GAAGTCCCTCACCCTCCCAA |
| | (SEQ ID #9) |
| m βactin R | GGCATGGACGCGACCA |
| | (SEQ ID #10) |

Adipogenesis

| | |
|---|---|
| m PPAR g F | TCGCTGATGCACTGCCTATG |
| | (SEQ ID #11) |
| m PPAR g R | GAGAGGTCCACAGAGCTGATT |
| | (SEQ ID #12) |
| m c/EBP a F | CAAGAACAGCAACGAGTACCG |
| | (SEQ ID #13) |
| m c/EBP a R | GTCACTGGTCAACTCCAGCAC |
| | (SEQ ID #14) |
| m FAS F | CTGAGATCCCAGCACTTCTTGA |
| | (SEQ ID #15) |
| m FAS R | GCCTCCGAAGCCAAATGAG |
| | (SEQ ID #16) |
| m AP2 F | CATGGCCAAGCCCAACAT |
| | (SEQ ID #17) |
| m AP2 R | CGCCCAGTTTGAAGGAAATC |
| | (SEQ ID #18) |

Results

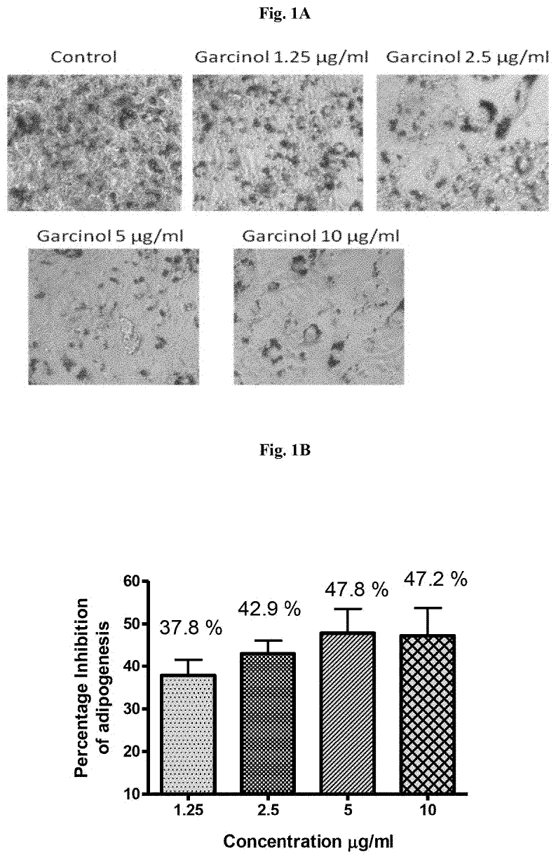

The lipids accumulated in adipocytes were quantified by Oil red O staining. Garcinol showed a dose dependent reduction in lipid accumulation in adipocytes (FIG. 1) with 5 and 10 μg/ml showing the highest inhibition of lipid accumulation by 47.8% and 47.2% (FIG. 1b).

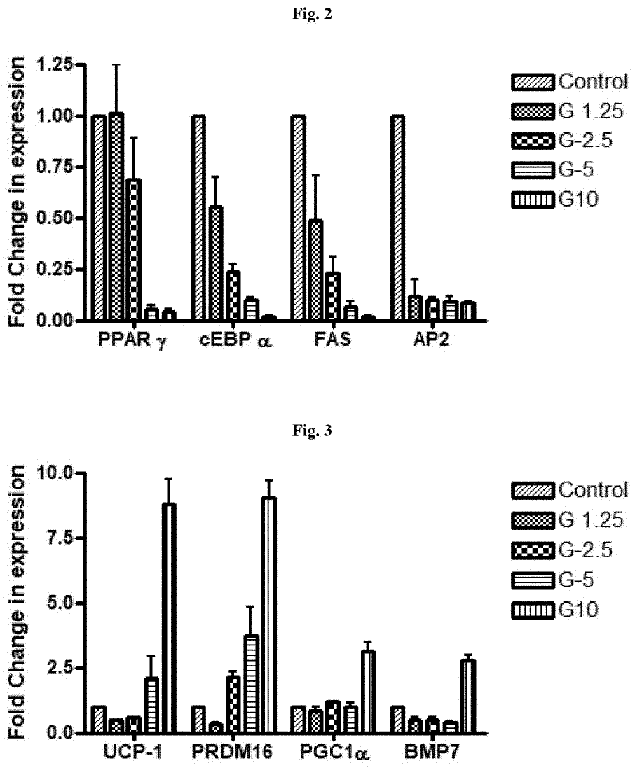

With respect to the genes involved in adipogenesis, PPARγ is considered to be the master regulator of adipogenesis. Decrease in PPARγ Expression will reduce the expression of other adipogenesis specific genes. In the present study, garcinol exhibited a dose expended reduction in the PPARγ Expression and the expression genes related to adipogenesis and fatty acid synthesis like cEBPα, FAS and AP2 (FIG. 2), indicating that garcinol inhibits adipogenesis in a dose dependent manner.

Garcinol also significantly increased the brown adipose tissue specific genes. The expression of UCP1, PRDM16, PGC1α and BMP7 was increased by garcinol in a dose dependent manner (FIG. 3) suggesting that garcinol is effective in converting the white adipose tissue depots to brown or brite/beigt adipose tissue thereby increasing energy expenditure by fat utilisation and lipolysis.

Effect of Garcinol on High Fat Diet Induced Obesity in C57 Mice

Methods

Animals—

C57/BL6 mice, 6-8 weeks of age and 8 animals/Group (4 Male and 4 Female) were used for the study. Animals were housed under standard laboratory conditions, air-conditioned with adequate fresh air supply (12-15 Air changes per hour), room temperature 20.2-23.5° C. and relative humidity 58-64% with 12 hours fluorescent light and 12 hours dark cycle. The temperature and relative humidity was recorded once daily.

Feed

The animals were fed with Normal diet (9 kcal/day) and High fat diet (50 kcal/day) throughout the acclimatization and experimental period.

Water was provided along with High Fat Diet to the animals throughout the acclimatization and experimental period. Water from water filter cum purifier was provided in animal feeding bottle with stainless steel sipper tubes.

All the studies were conducted according to the ethical guidelines of CPCSEA after obtaining necessary clearance from the committee (Approval No: 790/03/ac/CPCSEA).

a. In accordance with the recommendation of the Committee for the Purpose of Control and Supervision of Experiments on Animals (CPCSEA) guidelines for laboratory animal facility published in the gazette of India, Dec. 15, 1998.
b. The CPCSEA approval number for the present study (Anti-obesity activity) is SAC/IAEC/BC/2017/IP.-001.

The design of the study groups is depicted in table 2.

TABLE 2
Study design (16 weeks)

| Groups | Treatment | Diet |
|---|---|---|
| G1 | None | Normal diet (9 kcal % fat) |
| G2 | None | high fat diet (50 kcal % Fat) |
| G3 | Garcinol (5 mg/kgbw) | high fat diet (50 kcal % Fat) |
| G4 | Garcinol (10 mg/kgbw 16 | high fat diet (50 kcal % Fat) |
| | weeks. | |
| G5 | Garcinol (20 mg/kgbw) for | high fat diet (50 kcal % Fat) |
| | 16 weeks. | |
| G6 | Garcinol (40 mg/kgbw) | high fat diet (50 kcal % Fat) |

Body weight of the animals was recorded in all the days of experimental period. At the end of the experimental period, the animals were sacrificed by cervical dislocation. Blood was collected and Serum was separated by centrifugation and used for the analysis of biochemical parameters. The organs such as Liver Kidney, Spleen and Pancreas and Fat Tissues (Retroperitoneal, Peri-gonadal and Mesenteric) were dissected out and washed in phosphate buffered saline.

Efficacy Measurement

The following parameters were measured in the above groups:
Measurement of Body weight
Determination of Organ Weight
Estimation of Cholesterol (Zak et al., (2009) A new method for the determination of serum cholesterol. J Clin Endocrinol Metab., 94(7), 2215-2220)
Estimation of Triglycerides (Foster L. B and Dunn R. T. (1973) Stable reagents for determination of serum triglycerides by a colorimetric Hantzsch condensation method. Clin Chem. 196, 338-340).
Estimation of HDL Cholesterol (Burstein et al., (1970). Determination of HDL cholesterol. J. lipid Res., 11, 583).
Determination of LDL and VLDL (Friedewald et al., (1972) Estimation of the concentration of Low Density Lipoprotein cholesterol in plasma without use of preparative centrifuge. J. Clin Chem.; 18:499).

Results

Body Weight

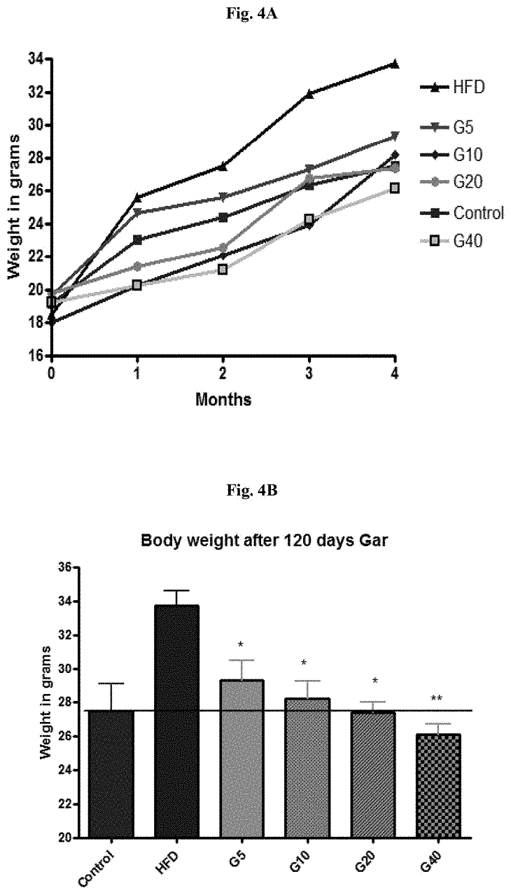

The results indicated that garcinol inhibited weight gain in a dose dependant manner in the animals fed with high fat diet (FIGS. 4a and 4b) for a period of 120 days. The percentage change in weight is depicted in the below table.

TABLE 3
Change in weight of a study animals

| | Control | HFD | G5 | G10 | G20 | G40 |
|---|---|---|---|---|---|---|
| Initial | 19.13 ± 0.91 | 18.50 ± 0.92 | 19.63 ± 0.94 | 18.00 ± 0.92 | 19.75 ± 0.88 | 19.25 ± 0.79 |
| weight (g) | | | | | | |
| Final | 27.5 ± 1.37 | 33.75 ± 1.60 | 29.33 ± 1.47 | 28.25 ± 1.39 | 27.37 ± 1.88 | 26.00 ± 1.33 |
| Weight (g) | | | | | | |
| Change in | 8.37 ± 1.41 | 15.25 ± 1.92 | 10.03 ± 2.25 | 10.25 ± 1.39 | 7.62 ± 2.18 | 6.28 ± 1.03 |
| weight (g) | | | | | | |

Reduction in Fat Deposits

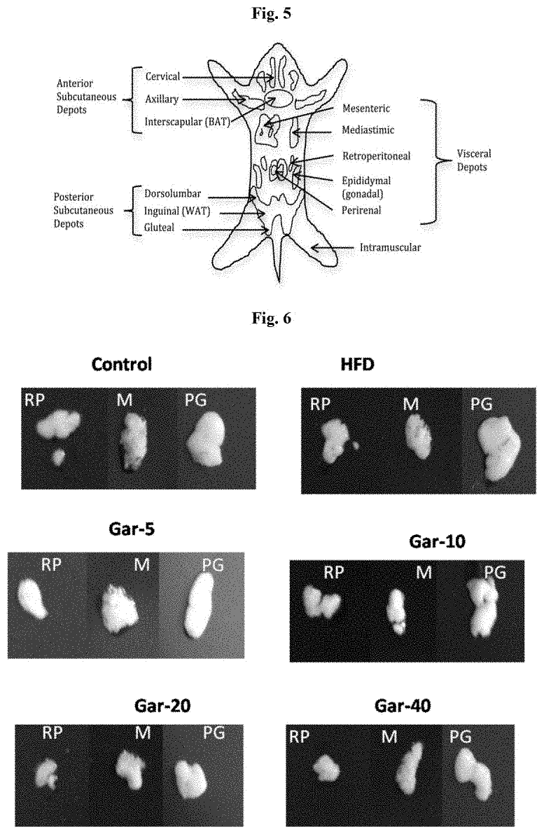

The fat reduction in the different fat pad regions of mice (FIG. 5) was also evaluated. The weights of Retroperitoneal, Peri-gonadal and Mesenteric Fat deposits after the 120 day administration of garcinol is tabulated as below

TABLE 4
Effect of Garcinol on Fat weight of HFD induced Mice

| | | Peri- | |
|---|---|---|---|
| | Retroperitoneal | gonadal Fat | Mesenteric |
| | Fat (g wet | (g wet | Fat |
| Groups | tissue) | tissue) | (g wet tissue) |
| I | 0.41 ± 0.03 | 1.14 ± 0.19 | 0.55 ± 0.03 |
| II | 0.55 ± 0.06 | 2.01 ± 0.22 | 0.69 ± 0.08 |
| III | 0.45 ± 0.06 | 1.33 ± 0.32 | 0.63 ± 0.07 |
| IV | 0.43 ± 0.05 | 1.17 ± 0.22 | 0.56 ± 0.05 |
| V | 0.46 ± 0.06 | 1.35 ± 0.21 | 0.601 ± 0.08 |
| VI | 0.46 ± 0.04 | 1.41 ± 0.14 | 0.59 ± 0.05 |

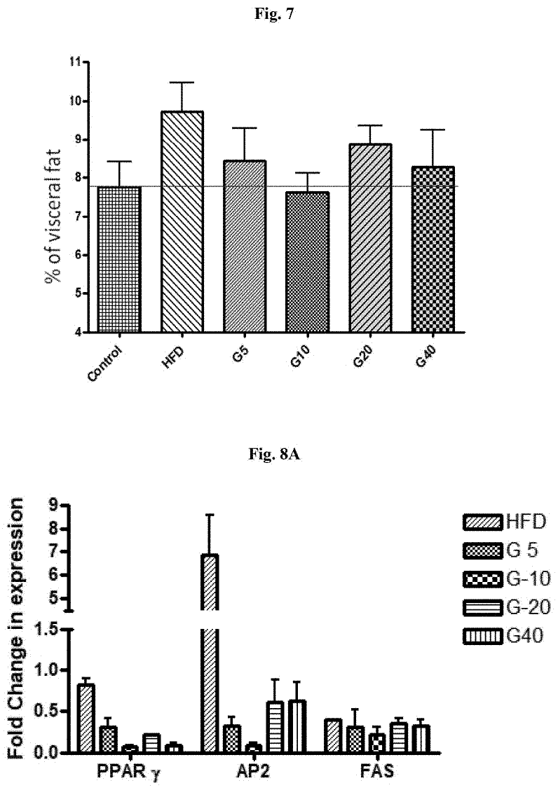

Garcinol treatment significantly reduced fat accumulation in the different fat pad regions (FIG. 6). Percentage of Visceral fat was reduced by garcinol treatment (FIG. 7) with the dose of 10 mg/kg body weight showing the maximum effect.

Organ Weights

Garcinol administration did not adversely affect the weight of the organs, suggesting that garcinol does not induce any adverse effects in critical organs. (Table 5).

TABLE 5
Weights of kidney, spleen and pancreas in garcinol treated animals

| | Kidney weight | Spleen Weight | Pancreas Weight |
|---|---|---|---|
| Groups | (g wet tissue) | (g wet tissue) | (g wet tissue) |
| I | 0.42 ± 0.02 | 0.19 ± 0.01 | 0.15 ± 0.01 |
| II | 0.553 ± 0.06 | 0.26 ± 0.03 | 0.24 ± 0.02 |
| III | 0.49 ± 0.03 | 0.25 ± 0.02 | 0.23 ± 0.01 |
| IV | 0.42 ± 0.03 | 0.20 ± 0.01 | 0.17 ± 0.01 |
| V | 0.45 ± 0.06 | 0.22 ± 0.03 | 0.21 ± 0.02 |
| VI | 0.42 ± 0.04 | 0.23 ± 0.02 | 0.22 ± 0.02 |

Gene Expression:

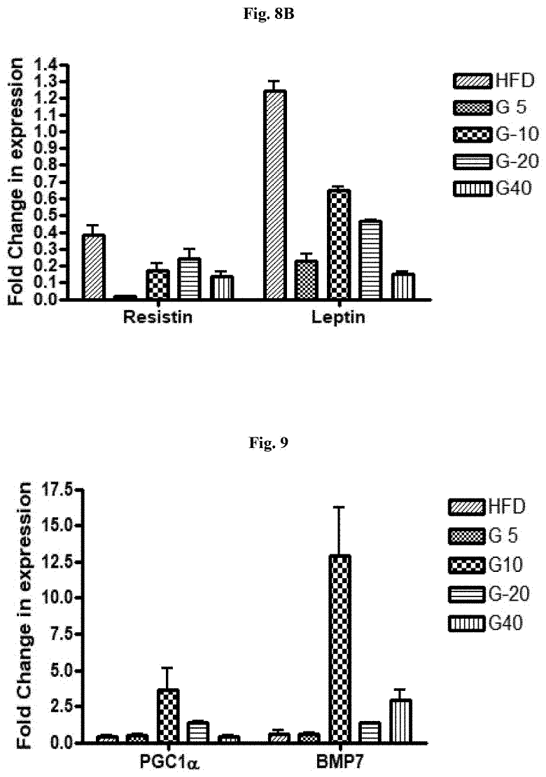

Reduction in the expression of genes related to adipogenesis was observed in fat pad of animals treated with Garcinol. Similar to Mouse 3T3-L1 cell lines, garcinol administration significantly reducted the expression of PPARγ, AP2, FAS, RESISTIN and LEPTIN in the fat pad regions (FIGS. 8a and 8b). Similarly, garcinol administration effectively increased the expression of Brown fat specific genes in the mice fat pad regions (FIG. 9).

Lipid Profile:

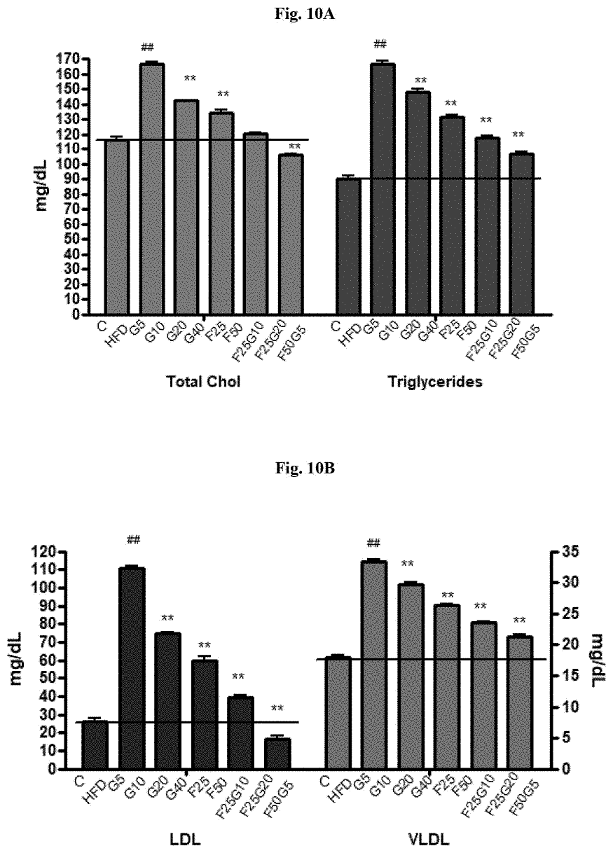

The high fat diet increased the levels of total cholesterol, LDL, VLDL and triglycerides in the serum of study animals. High fat diet, co administered with garcinol, significantly reduced the total cholesterol and triglycerides (FIG. 10a), LDL and VLDL (FIG. 10b) and increased the HDL levels (FIG. 10c) in the serum.

Conclusion:

Garcinol treatment showed a dose dependent inhibition of adipogenesis in vitro and induced the conversion of white adipose tissue to brown or brite/beige thereby increasing fat utilisation and energy metabolism. The in vivo results indicated that Garcinol was effective in significantly reducing body weight and visceral fat accumulation at 10 mg/kg and reduced adipogenesis specific gene expression and increased brown adipose tissue specific genes in fat pad in mouse fat pads. Garcinol administration also resulted in the reduction of visceral fat and organ weights indicating that garcinol promotes lipolysis and energy metabolism. Over all, garcinol induces weight loss, reduces viceral fat and maintains health of key organs.

### Example 2: Anti-Obesity Effects of Garcinol—Study Done at National Taiwan University, Taipei, Taiwan

Methodology

Reagents and Antibodies

AMPK and p-AMPK (Thr172) antibodies were purchased from Cell Signaling Technology (Beverly, Mass., USA). The SREBP-1 antibody was procured from Santa Cruz Biotechnology (Santa Cruz, Calif., USA). The PPARγ and Pref-1 antibodies were purchased from abcam (Cambridge, England). The mouse β-actin monoclonal antibody was obtained from Sigma Chemical Co (St. Louis, Mo., USA). The Bio-Rad protein assay dye reagent was purchased from Bio-Rad Laboratories (Munich, Germany). Xylenes and hematoxylin and eosin (H&E) stain were acquired from Surgipath (Peterborough, UK). Cholesterol used as part of the animal diet was obtained from Acros Organics (Bridgewater, N.J., USA). Garcinol was procured from Sabinsa Corp. (East Windsor, N.J., USA). The purity of garcinol was determined by high-performance liquid chromatography (HPLC) to be higher than 99%.

Animal Care and Experimental Design

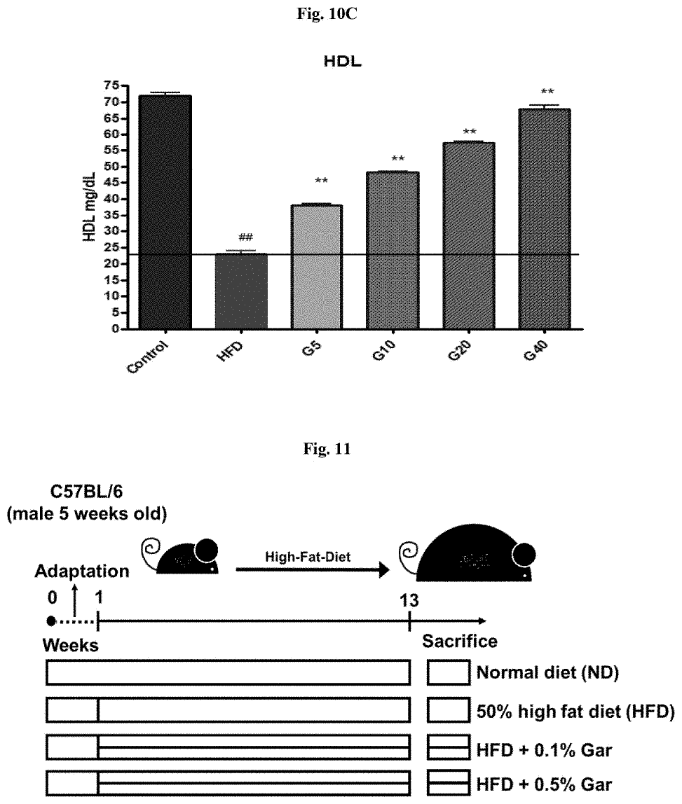

Five-week-old male C57BL/6 mice were purchased from the BioLASCO Experimental Animal Center (Taiwan Co., Ltd, Taipei, Taiwan) and housed in a controlled atmosphere (25±1° C. at 50% relative humidity) with a 12-h light/dark cycle. After one week of acclimation, animals were randomly distributed into normal diet (ND, 15% energy as fat), HFD (50% energy as fat), and HFD with 0.1% or 0.5% garcinol groups of eight mice in each group for 13 weeks. The experimental design is summarized in FIG. 11. The experimental diets were modified from the Purina 5001 diet (LabDiet, PMI Nutrition International, St. Louis, Mo., USA). The animals had ad libitum access to food and water. Food cups were replenished with a fresh diet every day. All animal experimental protocols employed in this study were approved by the Institutional Animal Care and Use Committee of the National Taiwan University (IACUC, NTU). Upon termination of the study, the animals were sacrificed by CO₂ asphyxiation and dissected, and the weights of their whole bodies and selected tissues, including the liver, kidney, spleen, adipose tissues (perigonadal, retroperitoneal, and mesenteric fat) and serum were immediately collected, weighed, and photographed.

Histopathological Examination

A portion of perigonadal fat and the median lobe of the liver were dissected and fixed in 10% buffered formalin, dehydrated with a sequence of ethanol solutions, and processed for embedding in paraffin. Sections of 3-5 μm in thickness were cut, deparaffinized, rehydrated, stained with H&E, and subjected to photomicroscopic assessment. Adipocyte size was determined using Image J software (Rasband, W. S., ImageJ, U.S. National Institutes of Health, Bethesda, Md., USA).

Biochemical Analysis

Blood samples were collected from the left ventricle under anesthesia. The samples were mixed in 10 μL of heparin sodium and centrifuged at 3500 rpm and 4° C. for 10 min. The plasma was then stored at −80° C. until use. Glutamic-pyruvic transaminase (GPT), total cholesterol (TC), TG, high-density lipoprotein (HDL), and low-density lipoprotein (LDL) levels were analyzed at the National Laboratory Animal Center, NLAC (Taipei, Taiwan) on a 7080 Biochemical Analyzer (Hitachi, Tokyo, Japan) according to the manufacturer's instructions.

16S rDNA Gene Sequencing and Analysis

Total DNA was extracted from fresh fecal samples. The purified DNA was eluted using the innuSPEED Stool DNA kit (Analytik Jena AG, Jena, Germany) according to the manufacturer's protocol. The PCR primer sequences from Caporaso et al., (Caporaso, J. G., Lauber, C. L., Walters, W. A., Berg-Lyons, D. et al., Global patterns of 16S rRNA diversity at a depth of millions of sequences per sample. Proc. Natl. Acad. Sci U.S.A. 2011, 108 Suppl 1, 4516-4522) were used to amplify the 16S rRNA variable region, and the PCR conditions were performed as mentioned in Tung et al., (Tung, Y. C., Lin, Y. H., Chen, H. J., Chou, S. C. et al., Piceatannol Exerts Anti-Obesity Effects in C57BL/6 Mice through Modulating Adipogenic Proteins and Gut Microbiota. Molecules. 2016, 21) and Chou et al., (Chou, Y. C., Suh, J. H., Wang, Y., Pahwa, M. et al. Boswellia serrata resin extract alleviates azoxymethane (AOM)/dextran sodium sulfate (DSS)-induced colon tumorigenesis. Mol. Nutr Food Res. 2017, 61) Then, the amplicons were used to construct index-labeled libraries with the Illumina DNA Library Preparation kit (Illumina, San Diego, Calif., USA). The Illumina MiniSeq NGS System (Illumina) was employed to analyze more than 100,000 reads with paired-end sequencing (2×150 bp), and the metagenomics workflow classified organisms from the amplicon using a database of 16S rRNA data. The classification was based on the Greengenes database (https://greengenes.lbl.gov/). The output of the workflow was a classification of reads at several taxonomic levels: kingdom, phylum, class, order, family, genus, and species.

Protein Preparation and Western Blot

Tissues were homogenized in ice-cold lysis buffer (10% glycerol, 1% TritonX-100, 1 mM Na₃VO₄, 1 mM EGTA, 10 mM NaF, 1 mM Na₄P₂O₇, 20 mM Tris buffer (pH7.9), 100 μM β-glycerophosphate, 137 mM NaCl, and 5 mM EDTA) containing 1 Protease Inhibitor Cocktail Tablet (Roche, Indianapolis, Ind., USA) on ice for 1 h, followed by centrifugation at 17,500 g for 30 min at 4° C. The protein concentration was measured with the Bio-Rad protein assay (Bio-Rad Laboratories, Inc., Hercules, Calif., USA).

Statistical Analysis

Statistical evaluation of the significance of the differences between the groups of mice was assessed using the Student t-test. For experiments comparing multiple groups, the differences were analyzed by carrying out one-way analysis of variance (ANOVA) and Duncan's post-hoc test. Data are presented as the means±SE for the indicated number of independently performed experiments, and p values <0.05 were considered statistically significant. Principal component analysis (PCA) was conducted to visualize the differences between samples.

Results

Body Weight Gain

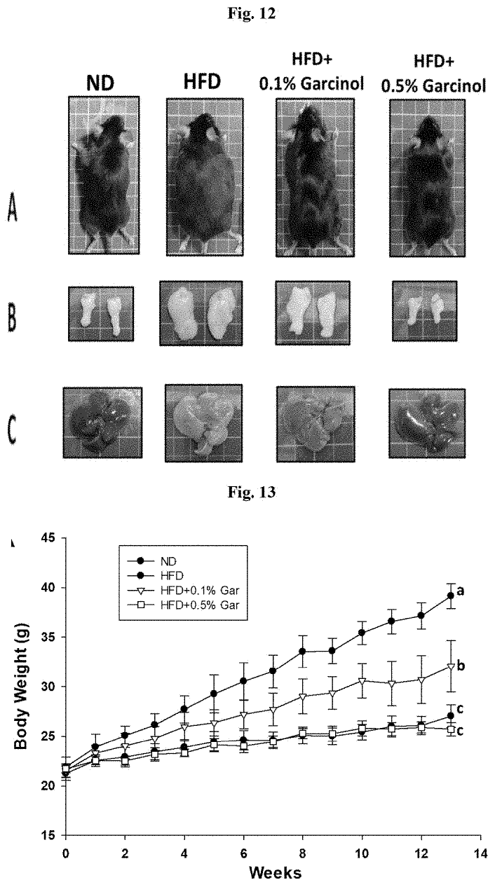

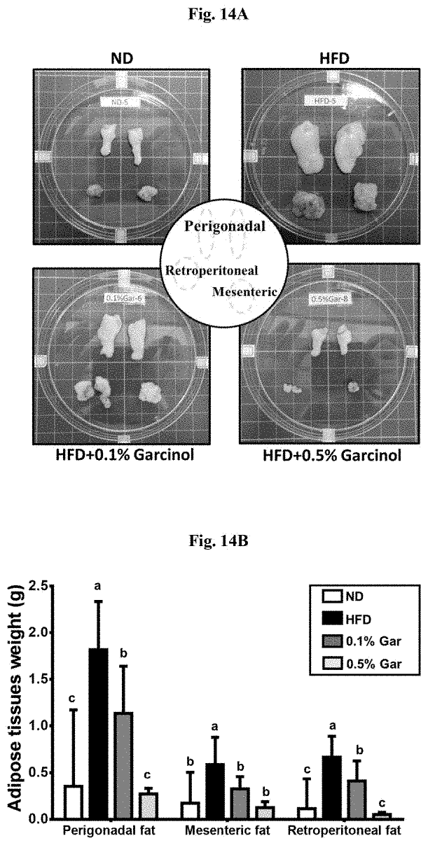

The results indicated that that HFD feeding for 13 weeks led to significant increase in body and liver weight along with perigonadal, retroperitoneal, and mesenteric fat accumulation. Diet supplemented with 0.1 and 0.5% garcinol reduced body weight in a dose-dependent manner. Co-treatment with high doses of garcinol (0.5%) and a HFD diet inhibited body weight gain, and there is no difference between HFD+0.5% garcinol and the ND group (FIG. 12 and FIG. 13).

Effect on White Adipose Tissue Adipocyte Size and Liver Homeostasis

Garcinol at concentrations of 0.5% dramatically decreased all three white adipose fat weights, compared to the HFD group, by 85.1% in terms of perigonadal weight, 92.4% retroperitoneal weight, and 77.7% mesenteric weight (FIGS. 14a and 14b).

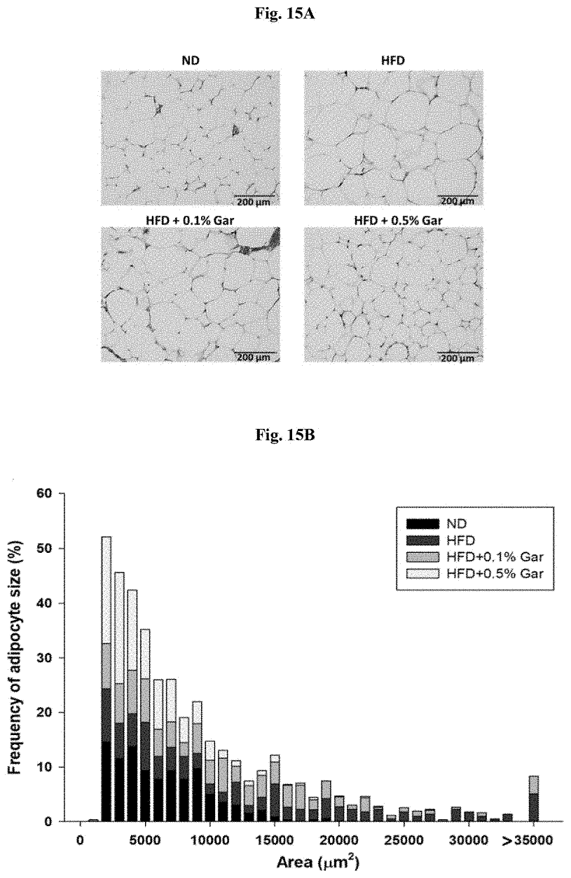

The average adipocyte size in perigonadal adipose tissue was evaluated by H&E staining, and the results revealed that adipocytes were enlarged in HFD-fed mice compared to those of ND mice. The increased adipocyte size was significantly reduced in the garcinol-treated mice (FIG. 15). Garcinol (0.5%) could prevent the enlargement of adipocytes induced by HFD, which made adipocyte distribution at a size of 2000 μm². Importantly, adipocyte size can be prevented or inhibited by garcinol in a dose-dependent fashion (Table 6).

TABLE 6
Effect of garcinol on adipocyte size

| | | | HFD + | HFD + |
|---|---|---|---|---|
| Adipocyte | | | 0.1% | 0.5% |
| Size area | ND | HFD | garcninol | garcinol |
| 2000 | 14.4 ± 2.6^{ab} | 9.84 ± 4.5^{b} | 8.38 ± 5.3^{b} | 19.2 ± 3.9^{a} |
| 15000 | 0.92 ± 0.4^{b} | 6.06 ± 2.5^{a} | 4.03 ± 1.0^{a} | 1.36 ± 0.9^{b} |
| >350000 | 0.00 ± 0.0^{c} | 5.10 ± 0.9^{a} | 3.32 ± 1.5^{b} | 0.00 ± 0.0^{c} |

The significance of the difference among the four groups was analyzed by one-way ANOVA and Duncan's multiple-range tests. The values with different letters are significantly different (p<0.05) between each group.

Lipid Profile: The plasma lipid profiles were also analyzed and are presented in Table 7.

TABLE 7
Lipid profile in mice adiministered with garcinol

| | ND | HFD | HFD + 0.1% Gar | HFD + 0.5% Gar |
|---|---|---|---|---|
| GPT (U/L) | 27.2 ± 7.04^{ab} | 32.1 ± 6.42^{a} | 20.5 ± 7.26^{b} | 27.6 ± 3.72^{ab} |
| T-CHO | 69.6 ± 7.31^{d} | 200.3 ± 11.30^{a} | 179.7 ± 11.85^{b} | 137.9 ± 11.78^{c} |
| (mg/dL) | | | | |
| TG (mg/dL) | 83.7 ± 14.56^{a} | 85.2 ± 13.09^{a} | 69.6 ± 19.90^{ab} | 55.6 ± 4.95^{b} |
| LDL (mg/dL) | 2.4 ± 0.41^{d} | 40.0 ± 2.89^{a} | 33.3 ± 0.72^{b} | 24.9 ± 4.47^{c} |
| HDL (mg/dL) | 57.8 ± 6.01^{c} | 155.6 ± 5.97^{a} | 152.6 ± 9.73^{a} | 118.7 ± 13.22^{b} |
| LDL/HDL | 0.04 ± 0.01^{c} | 0.25 ± 0.01^{a} | 0.22 ± 0.02^{b} | 0.21 ± 0.03^{b} |

Data are presented as the mean±SE. The significance of the differences among the four groups was analyzed by one-way ANOVA and Duncan's multiple-range tests. Values not sharing the same superscript letters in the same row are significantly different among groups. p<0.05; a, b, c, and d are significantly different between each group.

Mice administered with garcinol at 0.1% and 0.5% had significantly diminished serum levels of both TC and TG With respect to LDL and HDL, garcinol (0.1 and 0.5%) could reduce LDL levels, induced by HFD, in a dose-dependent manner. As the TC decrease was brought about by HFD, the HFD group increases not only the LDL levels, but also HDL levels. Hence, we used the LDL/HDL ratio to express this change. High and low dosages of garcinol can significantly diminish the LDL/HDL ratio compared to the HFD group.

Garcinol Reversed HFD-Induced Gut Dysbiosis

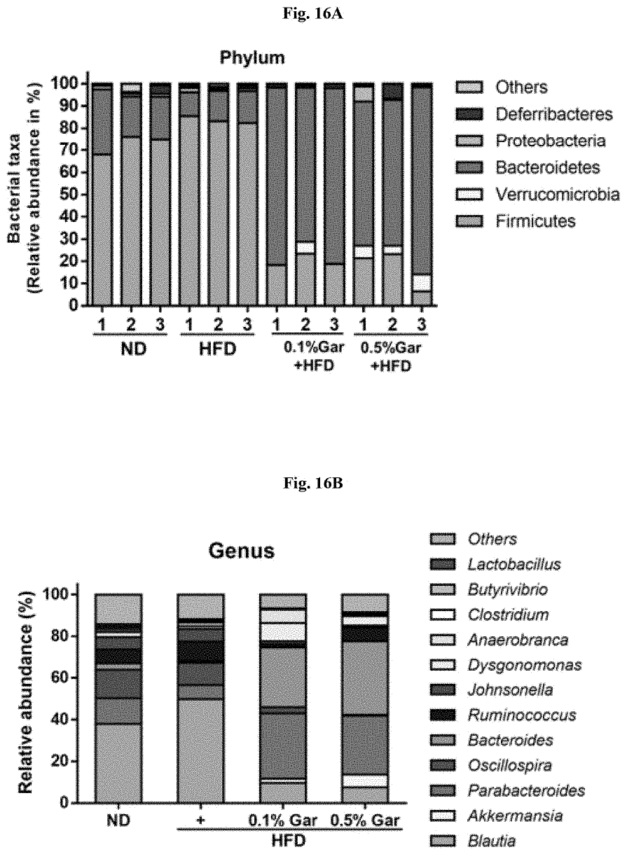

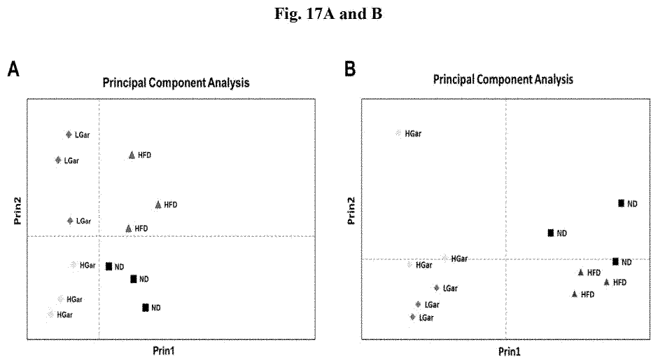

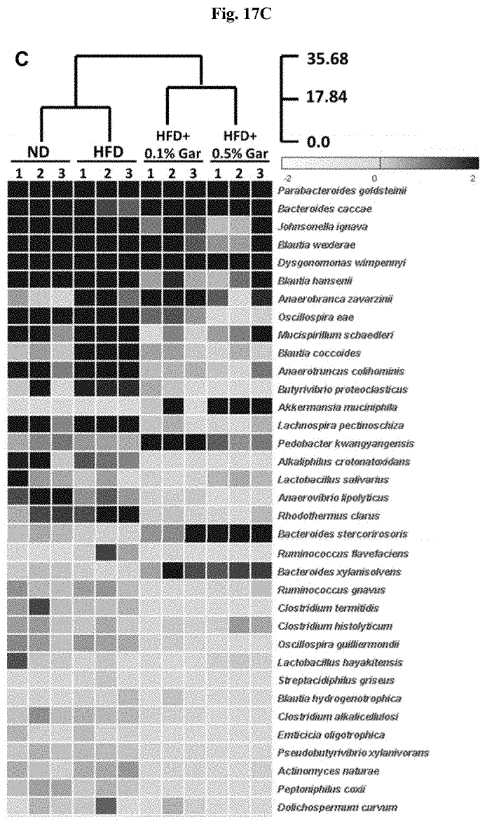

The overall composition of the bacterial community in the different groups was assessed by analyzing the degree of bacterial taxonomic similarity between metagenomic samples at the genus level. Bacterial communities were clustered using PCA, which distinguished microbial communities based on HFD diet/garcinol treatment. The gut microbiota of obese humans and HFD-fed mice is characterized by an increased Firmicutes-to-Bacteroidetes ratio (F/B ratio) (Brun, P., Castagliuolo, I., Di, L., V, Buda, A. et al., Increased intestinal permeability in obese mice: new evidence in the pathogenesis of nonalcoholic steatohepatitis. Am. J Physiol Gastrintest. Liver Physiol 2007, 292, G518-G525). The results indicated that the phylum level of HFD group has the higher F/B ratio compared with ND group (FIG. 16a). Interestingly, the Garcinol treatment reduced the F/B ratio by highly elevating Bacteroidetes communities. In addition, garcinol treatment made the Verrucomicrobia communities rise in number (FIG. 16b). PCA of UniFrac-based pairwise comparisons of community structures disclosed a distribution of the microbial community among the four groups of mice. The main finding of PCA was that different diets promoted the development of various gut microbial communities. HFD-fed mice formed a cluster that was distinct from ND group mice, and the HFD-fed mice were also distinct from garcinol treatment mice (FIGS. 17a, b and c). However, high doses of garcinol (0.5%) treated mice's microbial communities were closely clustered to that of ND mice, this indicates that garcinol has a marked effect on gut microbial community composition and also reversed HFD-induced gut dysbiosis.

Effects of Garcinol Administration on the Composition of Gut Microbial Communities

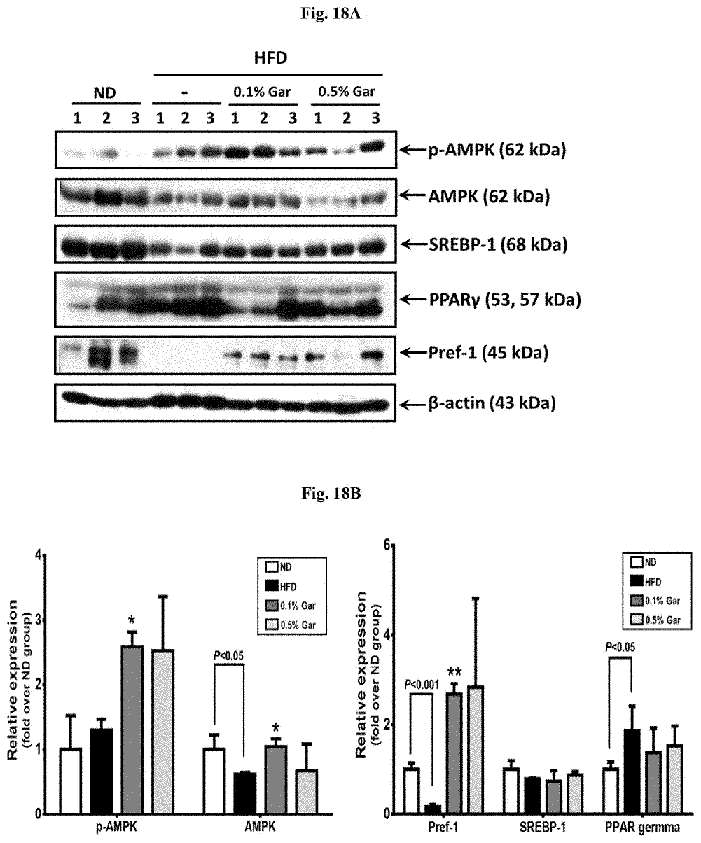

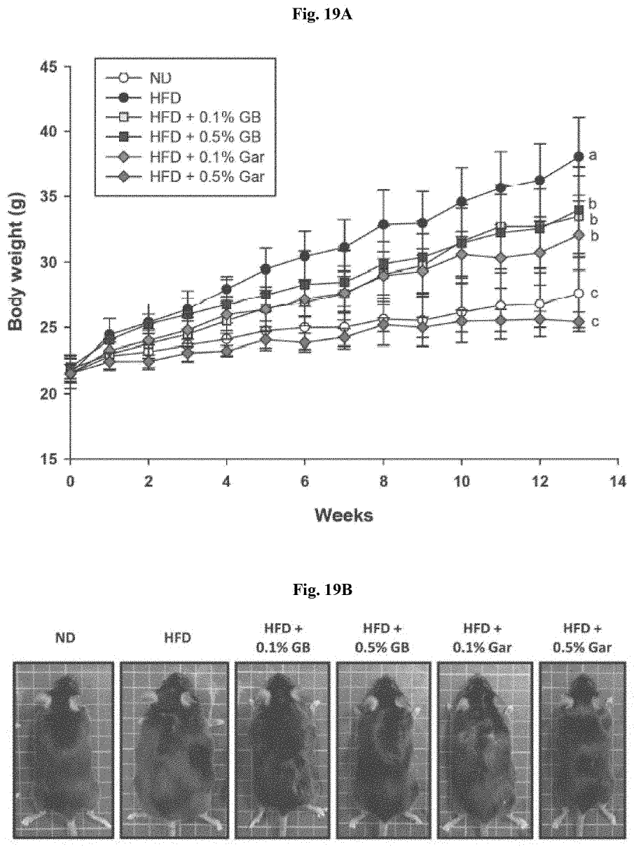

To further investigate whether the changes in the gut microbiota were induced by garcinol supplementation, we next determined the genus level of gut microbiota and used a heatmap to express the abundance of 50 OTUs significantly altered by garcinol in HFD-fed mice (FIG. 18). The results demonstrated that HFD-fed mice increased Blautia communities, which dramatically decreased in both high- and low-dose garcinol treatment groups. The studies pointed that Blautia spp. and Enterobacter spp. were related to a HFD causing obesity in a mouse model (Becker, N., Kunath, J., Loh, G, and Blaut, M. Human intestinal microbiota: characterization of a simplified and stable gnotobiotic rat model. Gut Microbes. 2011, 2, 25-33; Fei, N. and Zhao, L. An opportunistic pathogen isolated from the gut of an obese human causes obesity in germfree mice. ISME. J 2013, 7, 880-884). Interestingly, the Parabacteroides, Bacteroides, and Akkermansia genus also dramatically rose in number in the garcinol-fed mice. Parabacteroides and Bacteroides belong to the Bacteroidetes phylum, and Akkermansia belong to the Verrucomicrobia phylum; this explains why the F/B ratio behaved as it did following induction by garcinol treatment. In the heatmap, we observed that Anaerobranca zavarinii, Blautia coccoides, and Butyrivibrio proteoclasticus communities rose in number after HFD feeding, however garcinol administration not only lower those bacteria, but also Oscillospira eae, Mucispirillum schaedleri, Anaerotruncus colihominis, and Lachnospira pectinoschiza. In addition, garcinol increased the numbers of Akkermansia muciniphila, Bacteroides stercorirosoris, and Bacteroides xylanisolvens, which was diminished in the ND and HFD groups.

Anaerobranca zavarzinii, Blautia coccoides, and Butyrivibrio proteoclasticus belong to the Firmicutes phylum; Anaerobranca zavarzinii is positively correlated with IBD patients, and Blautia coccoides was increased in HFD-induced mice model. Butyrivibrio proteoclasticus is extremely sensitive to the toxic effects of unsaturated fatty acids and associated with obesity. On the other hand, Bacteroides stercorirosoris and Bacteroides xylanisolvens belong to the Bacteroidetes phylum, and Akkermansia muciniphila to the Verrucomicrobia phylum. Andoh et al. (Andoh, A., Nishida, A., Takahashi, K., Inatomi, O. et al., Comparison of the gut microbial community between obese and lean peoples using 16S gene sequencing in a Japanese population. J Clin. Biochem. Nutr 2016, 59, 65-70) performed 16S rRNA sequence analysis of the gut microbiota profiles of obese and lean Japanese populations, and they found that Bacteroides stercorirosoris exists in lean Japanese people. Liu et al. (Liu, R., Hong, J., Xu, X., Feng, Q. et al., Gut microbiome and serum metabolome alterations in obesity and after weight-loss intervention. Nat. Med 2017, 23, 859-868) performed a metagenome-wide association study and serum metabolomics profiling in lean and obese, young, Chinese individuals. They linked intestinal microbiota alterations with circulating amino acids and obesity, and indicated that Bacteroides xylanisolvens was significantly enriched in lean controls.

Several studies have highlighted the effects of the mucin-degrading bacterium, Akkermansia muciniphila, which is more abundant in the mucosa of healthy subjects than in that of diabetic patients or animals (Liou, A. P., Paziuk, M., Luevano, J. M., Jr., Machineni, S. et al., Conserved shifts in the gut microbiota due to gastric bypass reduce host weight and adiposity. Sci Transl. Med 2013, 5, 178ra41). Many studies have demonstrated the dietary effect of Akkermansia muciniphila and how it also inhibits obesity. Dietary supplementation of an HFD with grape polyphenols resulted in dramatic changes in the gut microbial community structure, including a reduction in the F/B ratio and a bloom of Akkermansia muciniphila (Roopchand, D. E., Carmody, R. N., Kuhn, P., Moskal, K. et al., Dietary Polyphenols Promote Growth of the Gut Bacterium Akkermansia muciniphila and Attenuate High-Fat Diet-Induced Metabolic Syndrome. Diabetes 2015, 64, 2847-2858). All these studies support the suggestion that Akkermansia muciniphila has a potential role as a probiotic with anti-obesity effects, therefore we suggest that garcinol exhibits the prebiotic role.

Garcinol Treatment Increased the Number of Akkermansia Spp. and Affected AMPK Signaling Pathway by Inducing Endocannabinoid Expression

We further investigated the molecular mechanisms by which garcinol exerts anti-obesity effects. The protein levels of AMPK, p-AMPK, PPARγ, preadipocyte factor 1 (Pref-1), and SREBP-1 in HFD-fed C57BL/6 mice are shown in FIG. 19. HFD feeding resulted in decreased AMPK compared to that of the ND group, but it was increased by administration of low doses of garcinol (0.1%) in white adipose tissue. Interestingly, high dosages of garcinol (0.5%) did not elevate AMPK protein or p-AMPK protein levels. We estimated this might be associated with Akkermansia spp. Administration of A. muciniphila to HFD-induced obese mice for four weeks improved endocannabinoid content (Everard, A., Belzer, C., Geurts, L., Ouwerkerk, J. P. et al., Cross-talk between Akkermansia muciniphila and intestinal epithelium controls diet-induced obesity. Proc. Natl. Acad. Sci U.S.A. 2013, 110, 9066-9071) including 2-AG, 2-PG, and 2-OG. Within intestinal tissue, the increase of 2-AG reduces metabolic endotoxemia and systemic inflammation by increasing goblet cell and Treg populations. However, in perigonadal adipose tissue, the increase of 2-AG also enhanced the storing capacity of adipose tissue by stimulating preadipocyte differentiation (via upregulation of adipocyte PPARγ levels), and enhancing de novo fatty acid synthesis (via stimulation of lipoprotein lipase and upregulation of FAS levels and glucose uptake), diminishing fatty acid oxidation (via inhibition of AMPK), and enhancing triacylglycerol accumulation (via inhibition of lipolysis). 2-AG is a phospholipid-derived lipid containing an arachidonic acid chain within its chemical structure. 2-AG is also an intermediate in triacylglycerol and phospholipid metabolism, so mice treated with HFD can readily supply the substrate for 2-AG production. Pref-1 is identified as an inhibitor of adipocyte differentiation that is highly expressed in preadipocytes and that disappears during differentiation. Garcinol treatment caused an increased protein level of Pref-1 in epididymal adipose tissue which suggests garcinol may function in the maintenance of the preadipose state in HFD-fed mice.

Conclusion

The results revealed that garcinol treatment brought about an unexpected change in the composition of the gut microbiota in mice receiving a HFD, which may affect the underlying molecular mechanisms. Moreover, these findings reinforce the concept that changes in the gut microbial community, with the goal of increasing the Akkermansia population, can prevent obesity induced by HFD.

### Example 3: Comparative Evaluation of Garcinol and Composition Containing Garcinol, Pterostilbene and Anthocyanin for Weight Loss

The present invention studied the anti-obesity effects of garcinol compared to a composition comprising garcinol, pterostilbene and anthocyanin (garcinol blend (GB) in mammals. The study was conducted in vivo on 5 weeks old C57BL/6 male mice. A total of 42 mice were involved in this study with 6 groups of 7 mice each. The groups were divided as in table 8.

The high fat diet (HFD) groups were fed 45% high fat diet for 16 weeks for the induction of obesity and concurrently administered the test substance as indicated in the aforesaid table. The normal group was fed with normal diet for 16 weeks.

TABLE 8
Study Groups

| Group | Diet | Test |
|---|---|---|
| 1 | Normal Diet | None |
| 2 | High Fat Diet (45%) | None |
| 3 | High Fat Diet (45%) | 0.1% GB |
| 4 | High Fat Diet (45%) | 0.5% GB |
| 5 | High Fat Diet (45%) | 0.1% Gar |
| 6 | High Fat Diet (45%) | 0.5% Gar |

Body weight was monitored weekly, and the average body weight of each group (n=7) was expressed as the means±SE. The significance of difference among the six groups was analyzed by one way ANOVA and Duncan's multiple range tests. p<0.05, a, b, and c significantly different between each group.

The results indicated that Mice fed with HFD+0.5% Gar groups showed the most significantly decreased body weight and prevented weight gain compared to the HFD fed group and HFD+GB group (FIGS. 19a and 19b). Mice administered with HFD+0.5% Gar showed the least weight gain compared to the other groups (Table 9) which is an unexpected finding and cannot be anticipated by a person skilled in the art.

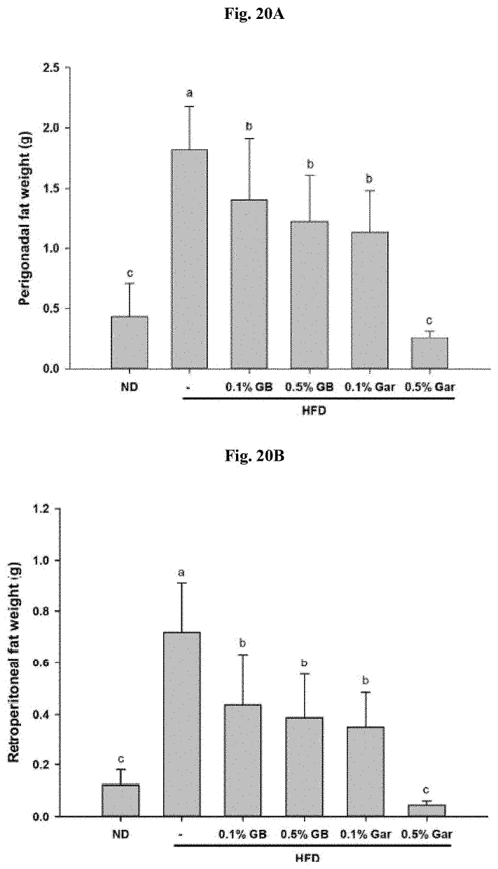

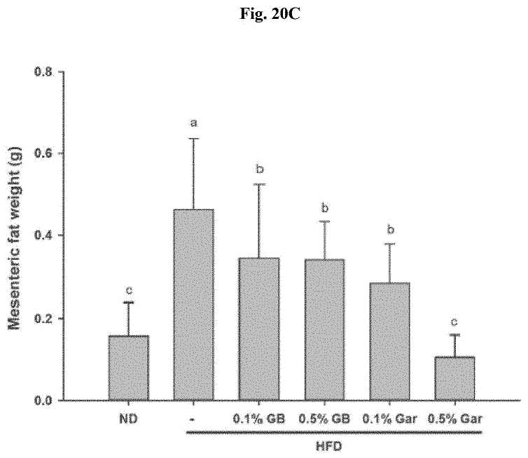

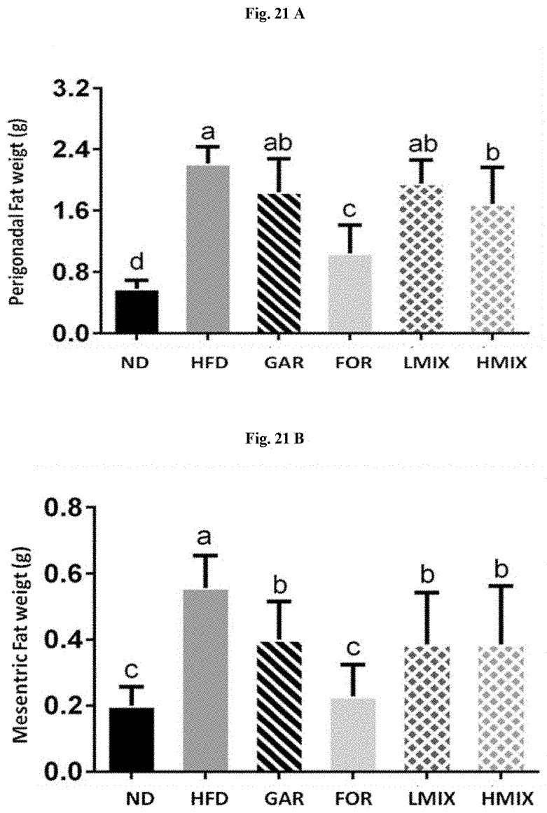

The effect of garcinol and garcinol blend on reducing the weight of perigonadal, retroperitoneal and mesenteric adipose tissues was also evaluated. The results indicated that 0.5% garcinol significantly reduced the weights of perigonadal, retroperitoneal and mesenteric adipose tissues compared to the garcinol blend (FIG. 20 a,b,c).

TABLE 9
Body weight of study animals administered with garcinol and garcinol
blend

| | | | HFD + | HFD + | HFD + | HFD + |
|---|---|---|---|---|---|---|
| | ND | HFD | 0.1% GB | 0.5% GB | 0.1% gar | 0.5% gar |
| Initial | 21.5 ± 1.1^{a} | 21.6 ± 1.1^{a} | 21.9 ± 1.0^{a} | 22.1 ± 1.0^{a} | 2.1.5 ± 0.7^{a} | 21.5 ± 0.7^{a} |
| weight (g) | | | | | | |
| Final | 27.7 ± 2.7^{c} | 38.1 ± 3.0^{a} | 33.5 ± 3.1^{b} | 34.0 ± 3.3^{b} | 32.1 ± 2.6^{b} | 25.4 ± 0.8^{b} |
| weight (g) | | | | | | |
| Weight | 6.1 ± 1.8^{c} | 16.5 ± 2.7^{a} | 11.6 ± 2.8^{b} | 11.9 ± 2.6^{b} | 10.5 ± 2.1^{b} | 3.9 ± 0.6^{b} |
| gain (g) | | | | | | |

Conclusion

Mice fed with HFD+0.5% garcinol showed significant reduction in weight compacted to the garcinol blend. This is an unexpected finding and cannot be anticipated by a person skilled in the art.

From the abovementioned examples, it is evident that garcinol brings about inhibition of adipogenesis and promotes weight loss in a dose dependant manner compared to the garcinol blend containing pterostilbene and anthocyanin. Garcinol also modifies the gut microbiota and increases the viable colonies of beneficial microbe—Akkermansia muciniphila thereby maintain and improving general health and well being. The present invention confirms that garcinol is an effective anti-obesity molecule and can be effective administered as a stand alone or in combination with other weight loss ingredients for the management of obesity and related disorders.

### Example 4: Comparative Evaluation of Garcinol (GAR), Forskolin (FOR) and Composition Comprising Garcinol and Forskolin at Low Dose (LMIX) and High Dose (HMIX) on Gut Microbiota in HFD-Fed Mice

Materials and Methods

Chemicals

Dulbecco's modified Eagle's medium (DMEM), penicillin-streptomycin, and fetal bovine serum (FBS) were obtained from Gibco BRL (Grand Island, N.Y., USA). Fetal calf serum was purchased from HyClone Laboratories (Logan, Utah, USA). Insulin, 3-isobutylmethylxanthine, dexamethasone, and Rosiglitazone were obtained from Sigma Chemical Co. (St. Louis, Mo., USA). Antibodies against PPARγ, C/EBPα, HSL, p-HSL, pPKA, and PPARα were purchased from Cell Signaling Technology (Beverly, Mass., USA) The antibody of CPT-1A were purchased from Abcam (Cambridge, England). Mouse β-actin monoclonal antibody was obtained from Sigma Chemical Co. (St. Louis, Mo., USA). Forskolin and Garcinol were also provided by Sami Labs Limited, and the purity of these compounds was above 95%.

Four-week-old, male C57BL/6 mice were purchased from the BioLASCO Experimental Animal Center (Taiwan Co., Ltd., Taipei, Taiwan) and housed in a controlled atmosphere (25±1° C. at 50% relative humidity) with a 12 h light/12 h dark cycle. After 1 week of acclimation, animals were randomly distributed into 6 groups as follows: normal diet (ND, 15% energy from fat), high-fat diet (HFD, 50% energy from lard-based fat), and HFD supplemented with 0.01% garcinol (GAR group), 0.025% Forskolin (FOR group), 0.005% GAR+0.025% FOR (LMIX group), and 0.01% Gar+0.025% For (HMIX group) for 12 weeks. The HFD were modified from the Purina 5001 diet as a normal diet, following our previous study (Lee et al., (2019). Garcinol Reduces Obesity in High-Fat-Diet-Fed Mice by Modulating Gut Microbiota Composition. Molecular nutrition & food research, 63(2), 1800390). Animals had free access to food and water. Food consumption was monitored daily and the body weight was recorded weekly. At the end of the experiments, all animals were sacrificed by CO₂ asphyxiation. Blood samples were collected from the heart for biochemical analysis. The liver, spleen, kidney, fat pads (perigonadal, retroperitoneal, and mesenteric fat), and fresh fecal pellets were immediately removed and stored at −80° C. until further analysis. All animal experimental protocols used in this study were approved by the Institutional Animal Care and Use Committee of the National Taiwan University (IACUC Approval No: NTU 06-EL-00114).

Histopathological Examinations

Liver and the perigonadal adipose tissue were dissected and fixed in 10% buffered formalin for at least 24 h, dehydrated with a sequence of ethanol solutions, and processed for embedding in paraffin. Sections of 4 μm in thickness were cut, deparaffinized, rehydrated, stained with hematoxylin and eosin (H&E), and subjected to photo microscopic observation. Adipocyte size was measured at 100× magnification and determined by Image J software (U. S. National Institutes of Health, Bethesda, Md., USA).

Western Blotting

Perigonadal tissues were homogenized with lysis buffer (50 mM Tris-HCl, pH 7.4; 1 mM NaF; 150 mM NaCl; 1 mM EGTA; 1 mM phenylmethanesulfonyl fluoride; 1% NP-40; and 10 μg/mL leupeptin) to extract total protein. The mixture then centrifuged at 17,000×g for 1 h at 4° C. The total protein content was measured by using the Bio-Rad Protein assay. Samples (50 μg of protein) were mixed with 5× sample buffer (0.3 M Tris-HCl (pH 6.8), 25% 2-mercaptoethanol, 12% sodium dodecyl sulfate (SDS), 25 mM EDTA, 20% glycerol, and 0.1% bromophenol blue). The mixtures were boiled at 100° C. for 10 min and subjected to 10% sodium dodecyl sulfate-polyacrylamide gelelectrophoresis (SDS-PAGE) at a constant current of 100 mA. After 3-4 h, the SDS-PAGE was transferred on the PVDF membrane with transfer buffer composed of 25 mM Tris-HCl (pH 8.9), 192 mM glycine, and 20% methanol. The membranes were blocked with blocking solution containing 20 mM Tris-HCl buffer with 1% of bovine serum albumin at room temperature for 1 h, then immunoblotted with primary antibodies against the target proteins and β-actin. The blots were rinsed with TPBS buffer (0.2% Tween 20 in 1×PBS buffer) for 10 min three times. Then, blots were incubated with a 1:5000 dilution of the horseradish peroxidase (HRP)-conjugated secondary antibody and then washed again three times with TPBS buffer. The transferred proteins were visualized with an enhanced chemiluminescence detection kit (ECL; Amersham Pharmacia Biotech, Buckinghamshire, UK).

Gut Microbiota Classification by Next Generation Sequencing (NGS)

Total DNA was extracted from fresh fecal samples by using the innuPREP Stool DNA kit (Analytik Jena AG, Jena, Germany) according to the manufacturer's instructions. According to the concentration, DNA was diluted to 1 ng μ L⁻¹ using sterile water. 6SrRNA/18SrRNA/ITS genes of distinct region (16SV4/16SV3/16SV3-V4/16SV4-V5, 18S V4/18S V9, ITS1/ITS2, Arc V4) were amplified using a specific primer (e.g. 16SV4: 515F-806R, 18S V4: 528F-706R, and 18S V9: 1380F-1510R) with a barcode. All PCR reactions were carried out using a Phusion® High-Fidelity PCR master mix (New England Biolabs, Ipswich, Mass., USA). The PCR products were mixed in ratios of equal density. Then, the mixed PCR products were purified using a Qiagen gel extraction kit (Qiagen, Hilden, Germany). Sequencing libraries were generated using the TruSeq® DNA PCR-free sample preparation kit (Illumina, San Diego, Calif., USA) following the manufacturer's recommendations, and index codes were added. The library quality was assessed using the Qubit@ 2.0 Fluorometer (Thermo Scientific) and Agilent Bioanalyzer 2100 system. Lastly, the library was sequenced using an Illumina HiSeq 2500 platform, and 250 bp paired-end reads were generated. The classification was based on the Greengenes database (https://greengenes.lbl.gov/).

Statistical Analysis

Data are represented as the means±SD for the indicated number of independently performed experiments. Comparisons of statistical significance between groups were made by one-way analysis of variance and Duncan's multiple range test SAS software (version 9.4, SAS Institute Inc., Cary, N.C., USA). A Student's paired test was performed to compare the significant differences between the treatment and control groups. A p-value<0.05 was considered statistically significant.

Results

Effects of GAR, FOR, LMIX, and HMIX on Body Weight, Food Intake, and Organ Weight in HFD-Induced Obese Mice

After the 12-week experimental period, the body weight of mice fed with the HFD was 37.4 g and significantly increased compared to mice fed with the ND (27.5 g). Final body weight significantly decreased in GAR (34.4 g), FOR (30.5 g), LMIX (34.8 g), and HMIX (33.8 g) groups compared to the HFD group. The weight gain of mice fed with the HFD was two folds compared to mice fed with ND. FOR had lowest weight gain than GAR, LMIX, and HMIX groups under no significant difference of food intake and food efficiency. GAR, FOR, LMIX, and HMIX significantly decreased liver weight, but no effects on kidney and spleen weight were observed. These results suggested that GAR, FOR, LMIX, and HMIX could ameliorate the process of additional energy stores in the body (Table 10).

TABLE 10
Effects of GAR, FOR, LMIX, and HMIX on Body Weight, Food
Intake and food efficiency

| Groups | ND | HFD | HFD + GAR | HFD + FOR | HFD + LMIX | HFD + HMIX |
|---|---|---|---|---|---|---|
| Initial | 21.0 ± 0.6^{a} | 21.5 ± 0.7^{a} | 21.5 ± 0.5^{a} | 21.7 ± 1.1^{a} | 21.1 ± 0.6^{a} | 21.8 ± 0.5^{a} |
| weight (g) | | | | | | |
| Final weight | 27.5 ± 0.5^{d} | 37.4 ± 1.4^{a} | 34.4 ± 2.6^{b} | 30.5 ± 2.6^{c} | 34.8 ± 2.4^{b} | 33.8 ± 3.0^{b} |
| (g) | | | | | | |
| Weight gain | 6.4 ± 0.9^{d} | 15.1 ± 1.7^{a} | 12.7 ± 2.0^{b} | 9.1 ± 1.8^{c} | 13.4 ± 1.8^{ab} | 12.0 ± 2.0^{b} |
| (g) | | | | | | |
| Food intake | 5.7 ± 0.2^{a} | 3.5 ± 0.1^{b} | 3.4 ± 0.1^{b} | 3.5 ± 0.1^{b} | 3.5 ± 0.2b | 35.0.2^{b} |
| (g) | | | | | | |
| Food | 1.4 ± 0.1^{b} | 4.8 ± 0.6^{a} | 4.3 ± 0.7^{a} | 3.6 ± 0.9^{a} | 4.7 ± 0.2a | 4.3 ± 0.9^{a} |
| efficiency | | | | | | |
| (g/day/mice) | | | | | | |
| Liver (g) | 1.4 ± 0.1^{c} | 1.9 ± 0.1^{a} | 1.6 ± 0.2^{b} | 1.5 ± 0.2^{bc} | 1.6 ± 0.2b | 1.6 ± 0.3^{b} |
| Kidney (g) | 0.4 ± 0.05^{a} | 0.4 ± 0.03^{a} | 0.4 ± 0.03^{a} | 0.4 ± 0.02^{a} | 0.4 ± 0.02a | 0.4 ± 0.03^{a} |
| Spleen (g) | 0.06 ± 0.02^{a} | 0.07 ± 0.01^{a} | 0.06 ± 0.01^{a} | 0.06 ± 0.01^{a} | 0.06 ± 0.01a | 0.07 ± 0.01^{a} |

Effects of GAR FOR, LMIX, and HMIX on Adipose Tissue and Liver in HFD-Induced Obese Mice

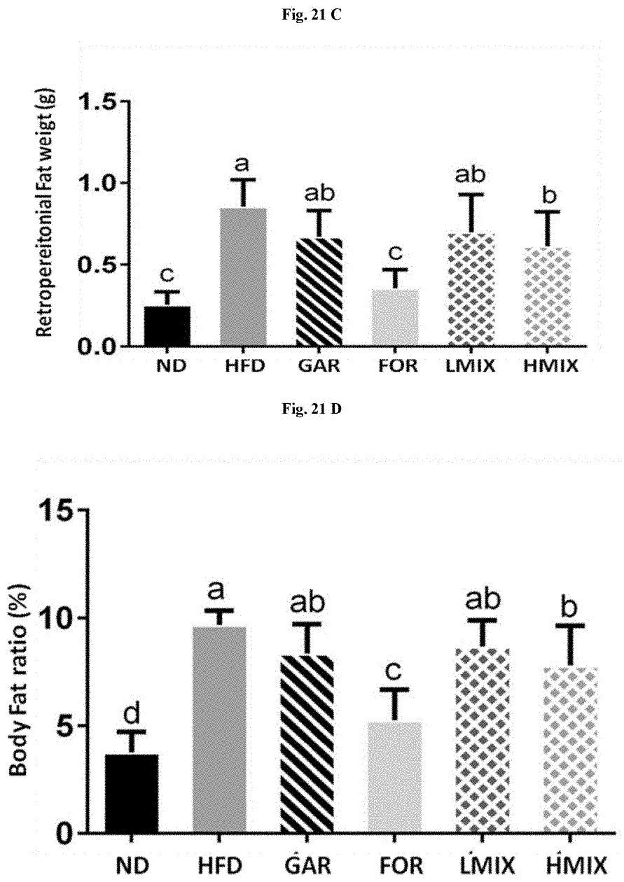

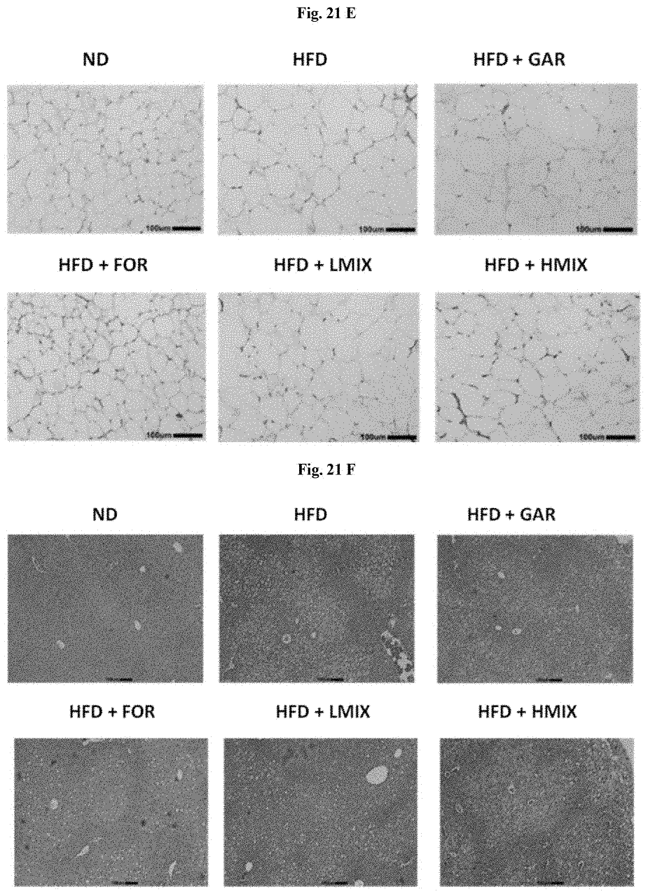

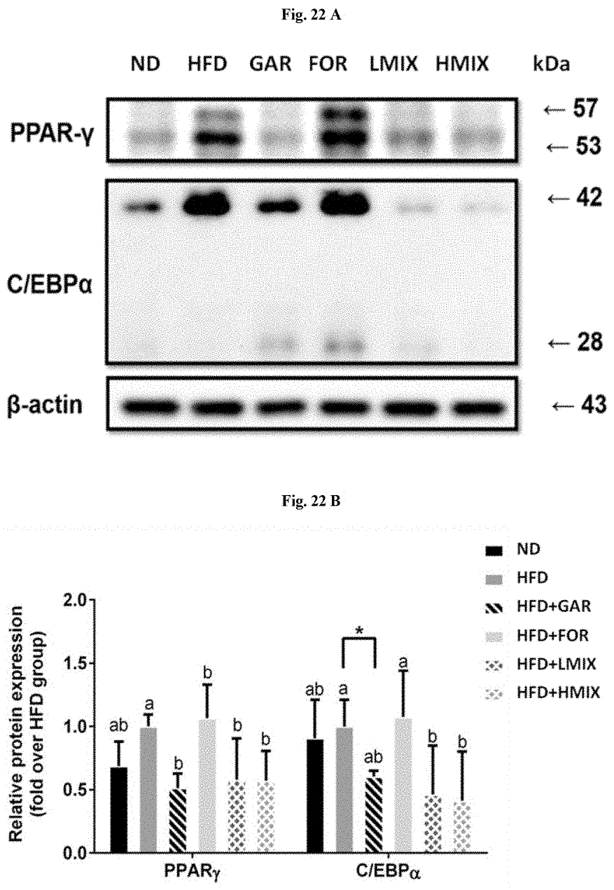

GAR, FOR, LMIX, and HMIX groups significantly decreased the weight of mesenteric fat, and FOR and HMIX further significantly decreased the weight of perigonadal and retroperitoneal fat compared with the HFD group. Overall, FOR and HMIX had a lower body fat ratio than other groups (FIG. 21A-21D). Furthermore, we examined the adipocyte size of perigonadal fat, with the results showing that GAR, FOR, LMIX, and HMIX groups significantly decreased adipocyte size, with the adipocyte size of the FOR group being almost as small as the ND group (FIG. 21E). On the other hand, a significant reduction in liver fat accumulation was observed after supplementation of FOR compared with HFD group (FIG. 21F).

Effects of GAR, FOR, LMIX, and HMIX on Adipogenesis, Lipolysis, and β-Oxidation Pathways in HFD-Induced Obese Mice

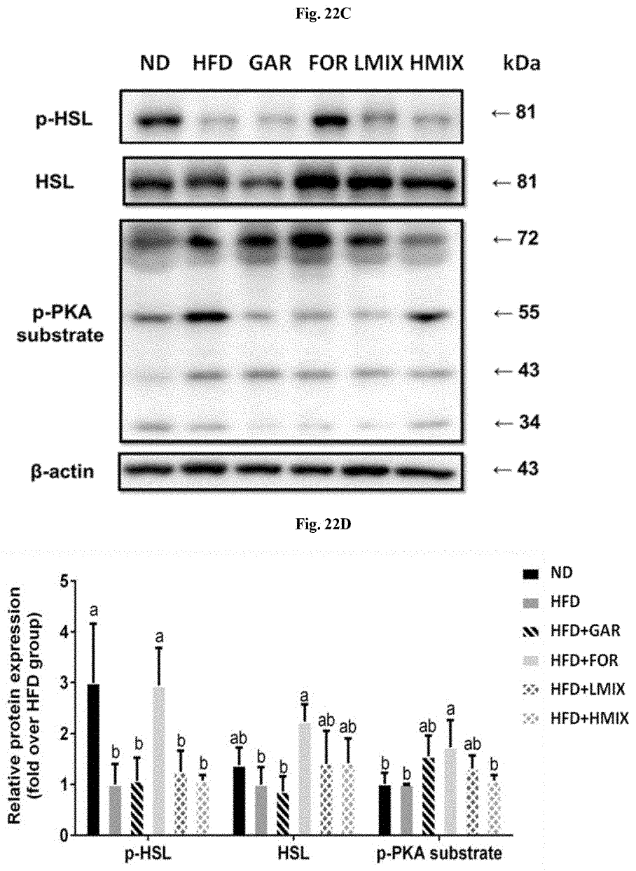

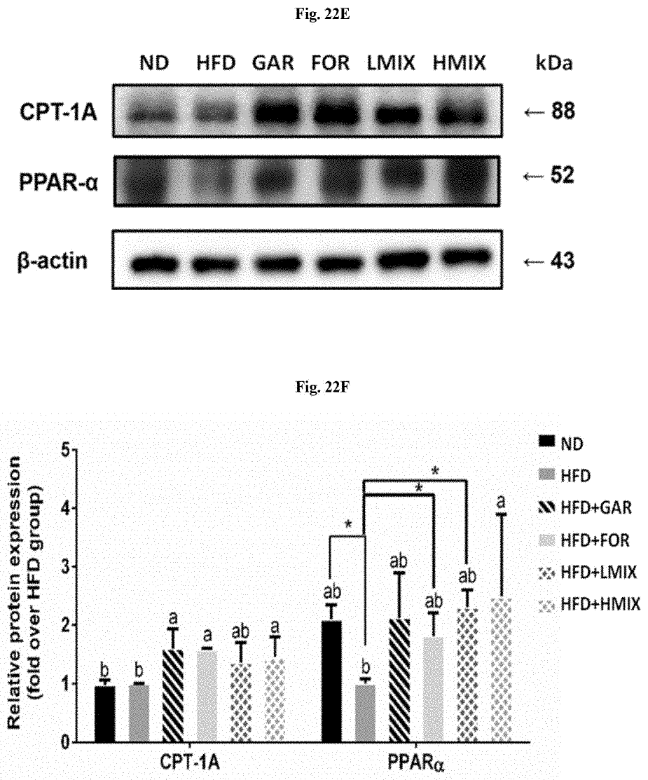

We further performed western blot analysis to investigate the related protein expression of adipogenesis, lipolysis, and β-oxidation in perigonadal adipose tissue. Although there was no significant difference in PPARγ and CIEBPα protein expression between HFD and ND groups, ND had lower PPARγ and C/EBPα protein expression. Further, protein expression of PPARγ and C/EBPα was significantly decreased after supplementation of GAR, LMIX, and HMIX compared to the HFD group, except for the FOR group (FIGS. 22A and 22B). This result indicated that GAR, LMIX, and MIX supplementations attenuated lipid accumulation through the regulation of adipogenesis-related protein expression. We proceeded to evaluate the related protein expression of the lipolysis pathway. GAR, LMIX, and HMIX groups showed no significant difference in p-HSL and p-PKA substrate protein expression compared to the HFD group (FIGS. 22C and 22D). Also, up regulated protein levels of p-PKA substrate and p-HSL were found in the FOR group compared to the HFD group, indicating that FOR could activate HSL to promote lipolysis in mice fed with HFD to attenuate adiposity. Although there was no significant difference in the CPT-1A protein expression between HFD and ND groups, the protein expression of CPT-1A was significantly increased after supplementation of GAR, FOR, and HMIX compared to the HFD group. Although there was no significant difference between LMIX and HFD groups, the CPT-1A expression of the LMIX group still had an upward trend. In addition, the protein expression of PPARα was increased after supplementation of GAR, FOR, LMIX, and HMIX groups compared to the HFD group (FIGS. 22E and 22F).

Effects of GAR, FOR, LMIX, and HMIX on Gut Microbiota in HFD-Fed Mice

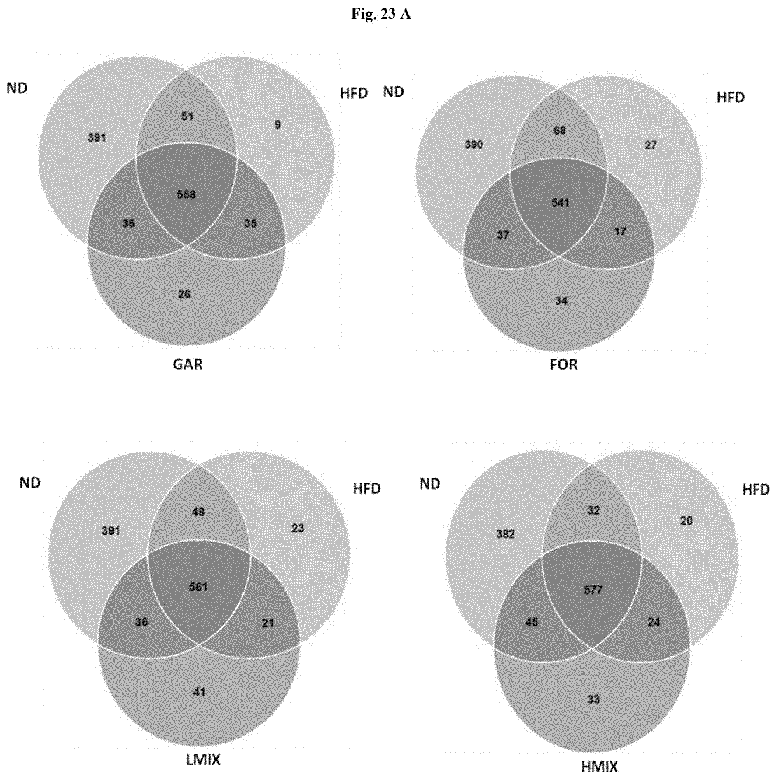

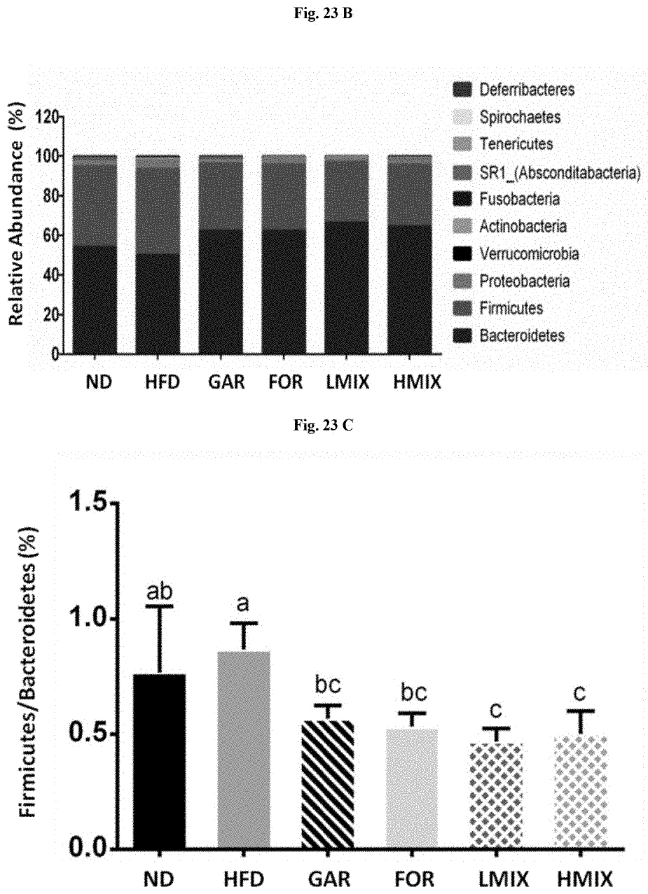

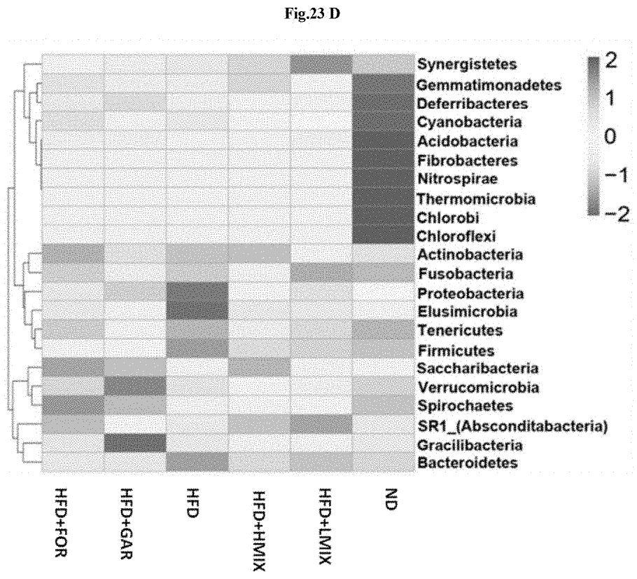

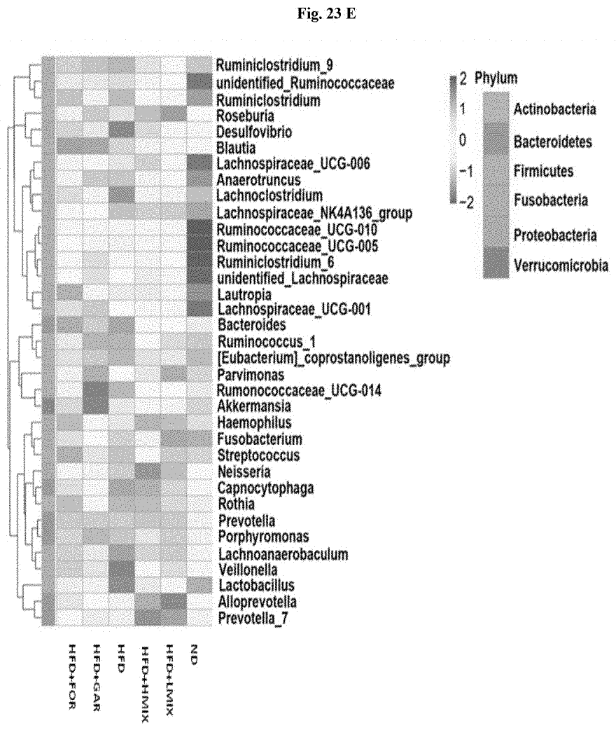

Consumption of a high-fat diet for 12 weeks altered the composition of the gut microbiota. According to a Venn diagram, GAR had 26 OTUs, FOR had 34 OTUs, LMIX had 41 OTUs, and HMIX had 33 OTUs difference between ND and HFD groups (FIG. 23A).

Bacteroidetes and Firmicutes are the most abundant groups of microbes present in the gut (Ley et al., Human gut microbes associated with obesity (2006) Nature, 444; 1022-1023). The levels of these microbes play a major role in the development and etiology of many diseases and disorders. Generally diet plays a major role in the diversity of gut microbiota. Individuals consuming foods rich in insoluble carbohydrates will have elevated levels of Firmicutes in their gut. These carbohydrate fermenters help in increased efficiency of energy salvage from food and thereby contribute to the weight gain (Balakrishnan S Ramakrishna, Role of the gut microbiota in human nutrition and metabolism, Journal of Gastroenterology and Hepatology 2013; 28 (Suppl. 4): 9-17). Firmicutes are also involved in the release of short chain fatty acids which are essential for maintaining healthy brain function. A study by Huang et al. (Huang et al. Possible association of Firmicutes in the gut microbiota of patients with major depressive disorder, Neuropsychiatr Dis Treat. 2018; 14: 3329-3337), reported a negative correlation between the level of Firmicutes and major depressive disorder.

Bacteroidetes, predominantly participate in carbohydrate and fat metabolism by expressing enzymes such as glycosyl transferases, glycoside hydrolases and polysaccharide lyases. The viable counts of these groups of microbes are evidently lower in obese individuals and increasing the levels of Bacteroidetes, help in reducing weight and increasing lean body mass. Lower levels of Bacteroidetes are also reported to be associated with the development of Inflammatory Bowel Disease (Zhou et. al., Lower Level of Bacteroides in the Gut Microbiota Is Associated with Inflammatory Bowel Disease: A Meta-Analysis, BioMed Research International, 2016, Article ID 5828959, 9 pages, http://dx.doi.org/10.1155/2016/5828959). Their levels are also reported to be lower in individuals with Non-alcoholic steato hepatitis (NASH). Thus, both Firmicutes and Bacteroidetes play an important role in maintaining normal health and metabolism. An elevated portion of Firmicutes and decreased levels of Bacteroidetes have been reported in obese patients (Ley et al., Human gut microbes associated with obesity (2006) Nature, 444; 1022-1023). A decrease in the ratio of Firmicutes/Bacteroidetes is also directly related to weight loss. Hence, any compound that modifies the Firmicutes/Bacteroidetes ratio may be used as an effective anti-obesity molecule.

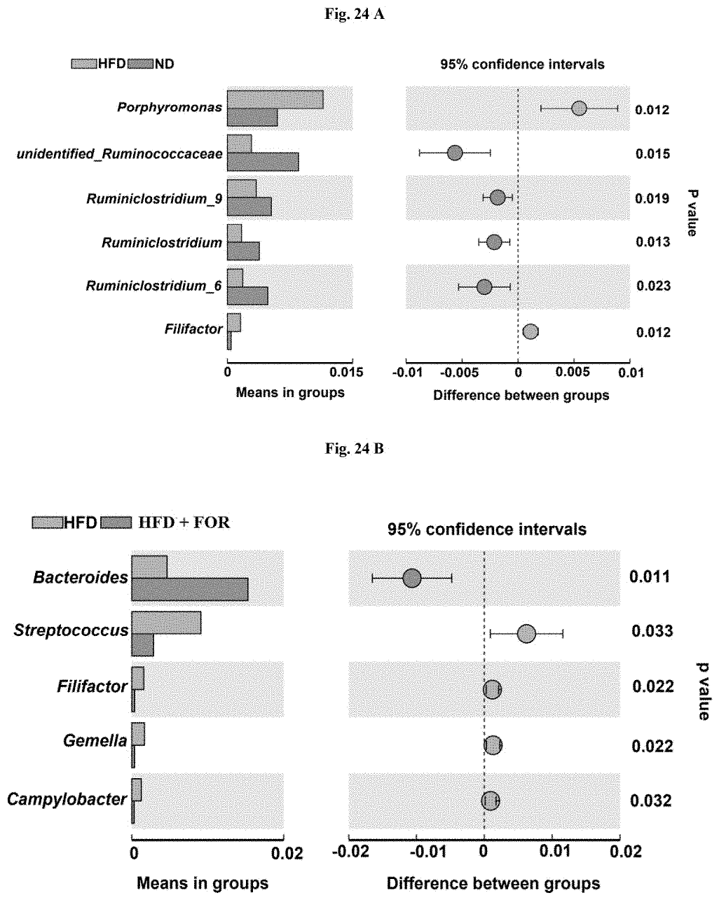

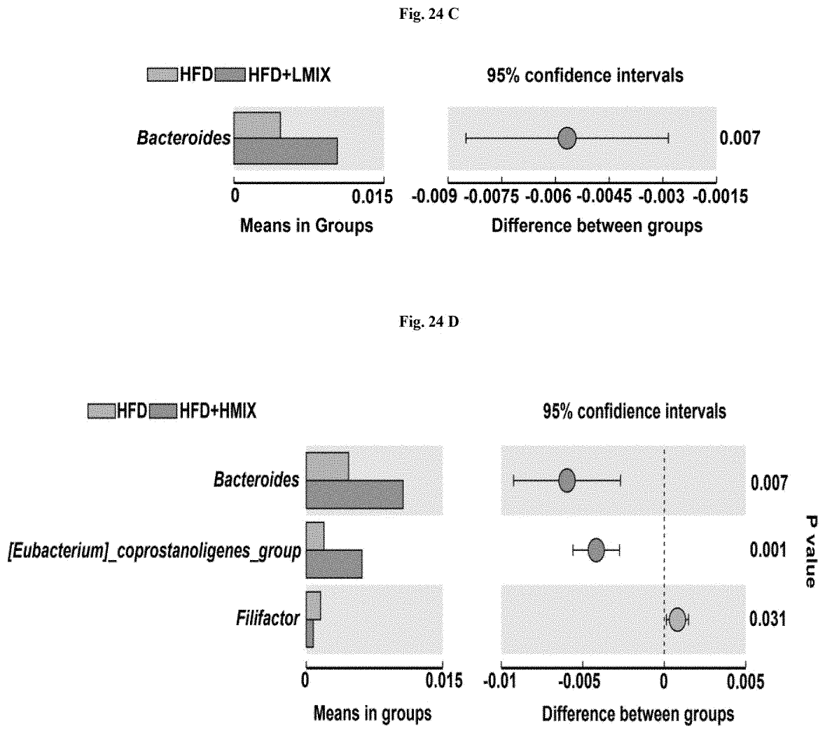

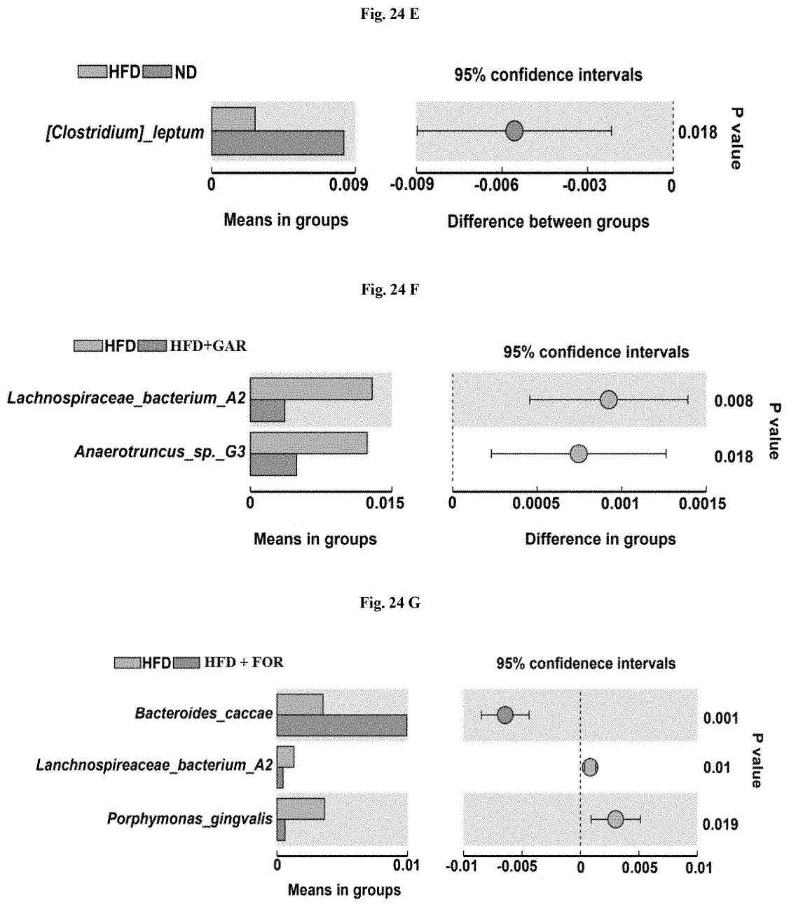

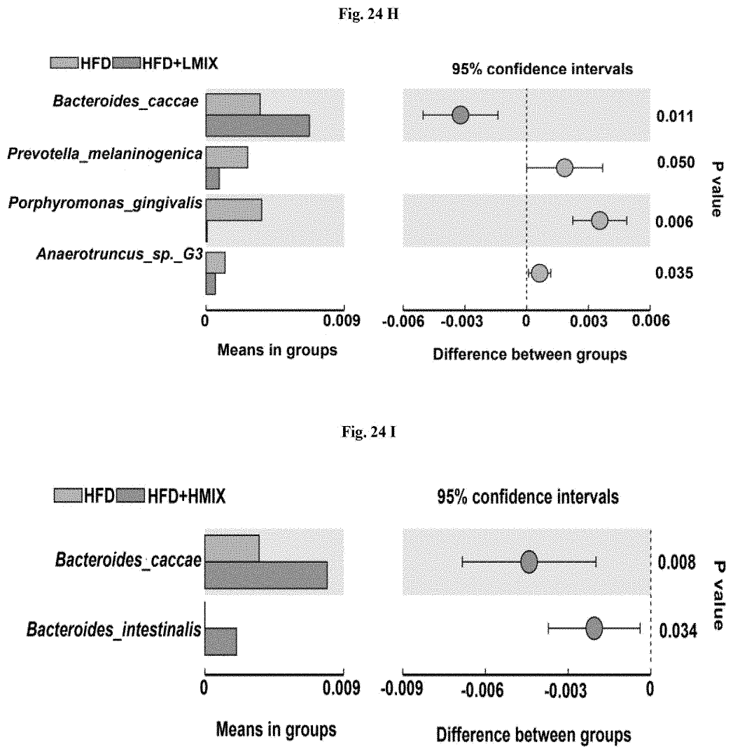

In the present invention, the abundance of Firmicute in the HFD group was 43.0%, which was higher than the 40.8% observed in the ND group. Mice fed with GAR, FOR, LMIX, and HMIX groups ameliorated the decline of the abundance of Firmicutes to 33.9%, 33.5%, 30.5%, and 31.1%, respectively, compared with the HFD group. For the other major bacterial phylum in the gut, Bacteroidetes, the abundance was 50.2% in the HFD group, a result which was lower than the 54.0/for the ND group. Supplementation of GAR, FOR, LMIX, and HMIX increased the abundance of Bacteroidetes to 62.4%, 62.2%, 66.4%, and 64.5%, respectively, compare with HFD. GAR, FOR, LMIX, and HMIX groups had a significantly lower F/B ratio than the HFD group (FIGS. 23B and 23C). The heat map revealed the abundance of the top 35 bacteria across the groups at the phylum and genus level. In the phylum level, the ND group had more relative abundance of gemmatimonadetes, deferribacteres, cyanobacteria, acidobacteria, fibrobacteres, nitrospirae, thermomicrobia, chlorobi, and chloroflexi among other groups. The HFD group had more abundance of proteobacteria, elusimicrobia, and Firmicutes and a lower relative abundance of Bacteroidetes among other groups. GAR group had more abundance of gracilibacteria and verrucomicrobia among other groups. The LMIX group had more relative abundance of synergistetes among other groups (FIG. 23D). In the genus level, the ND group had more relative abundance of lachnospiraceae_UCG-006, lachnospiraceae_UCG-006, ruminococcaceae_UCG-010, ruminococcaceae_UCG-005, and ruminococcaceae_6, all of which belong to the phylum firmicute. The HFD group had more abundance of desulfovibrio, lachnoclostridium, veillonella, and lactobacillus. The GAR group demonstrated abundance of rumonococcaceae_UCG-001 and Akkermansia. FOR had more relative abundance of blautia and bacteroides, whereas LMIX had increased relative abundance of roseburia. The HMIX group had more relative abundance of neisseria, alloprevotella, and prevotella_7 (FIG. 23E). In the genus level, the HFD group significantly had more abundance of Porphyomonas and Filifactor, and less abundance of Ruminiclostrium, Ruminiclostridium_6, and Ruminiclostridium_9 than ND. The FOR group significantly had more abundance of Bacteroides, and less abundance of Streprococcus, Filitactor, Gemelli, and Campylobacter than the HFD group. The LMIX group significantly had more abundance of Bacteroides than the HFD group, whereas HMIX significantly had more abundance of Bacteroides and [Eubacterium]_coprostanoligenes_group, and less abundance of Filitactor than the HFD group (FIG. 24A-24D). In the species level, the HFD group significantly had more abundance of [Clostridium] leptum than the ND group; the GAR group significantly had less abundance of Lachnospiraceae_bacterium_A2 and anaerotruncus_sp._G3 than the HFD group. The FOR group significantly had more abundance of Bacteroides caccae and less abundance of Lachnospiraceae-bacterium_A2 and Porphymonas-gingvalis than the HFD group. The LMIX group significantly had more abundance of Bacteroides_caccae, and less abundance of Prevotella_melaningogenica, Porphymona_gingvalis and anaerotruncus_sp._G3 than the HFD group. The HMIX group significantly had more abundance of Bacteroides_caccae, and Bacteroides_intestinalis than the HFD group (FIG. 24E-241). These results showed that GAR, FOR, LMIX, and HMIX groups modulated the gut flora that were altered by HFD.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method of modifying the gut microbial diversity in mammals, comprising step of administering effective amounts of a composition comprising garcinol, individually or in combination with forskolin, to said mammals, to bring about change in the gut microbial diversity.

2. The method as in claim 1, wherein the phylum of gut microbiota is selected from the group comprising of Deferribacteres, Proteobacteria, Bacteroidetes, Verrucomicrobia Firmicutes, Actinobacteria and Fusobacteria.

3. The phylum of gut microbiota as mentioned in claim 2, wherein the composition comprising garcinol, individually or in combination with forskolin decreases Firmicutes/Bacteroidetes ratio.

4. The phylum of gut microbiota as mentioned in claim 2, wherein the genus of the gut microbiota is selected from the group consisting of Lactobacillus, Butyrivibrio, Clostridium, Anaerobranca, Dysgonomonas, Johnsonella, Ruminococcus, Bacteroides, Oscillospira, Parabacterroides, Akkermanisa, Blautia, Neisseria, Synergistetes, Gemmatimonadetes, Cyanobacteria, Acidobacteria, Fibrobacteres, Nitrrospirae, Thermomicrobia, Chlorobi, Chloroflexi, Actinobacteria, Fusobacteria, Elusimicribia, Tenericutes, Firmicutes, Saccharibacteria, Verrucomicrobia, Sphrochaetes, Absconditabacteria, Gracillibacteria, Bacteriodetes, Ruminoclostridium, Roseburia, Desulfovibrio, Anaerotruncus, Lautropia, Rumonococcaceae, Haeemphilus, Capnocytophaga, rothia, Prevotella, Porphyromonas, Lachnoanaerobaculum, Veillonella and Alloprevotella.

5. The phylum of genus of gut microbiota as mentioned in claim 4, wherein the gut microbial species is selected from the group consisting of Parabacteroides goldsteinii, Bacteroides caccae, Johnsonella ignava, Blautia wexlerae, Dysgonomonas wimpennyi, Blautia hansenni, Anaerobranca zavarzinni, Oscillospira eae, Mucispirillus schaedleri, Blautia coccoides, Anaerotruncus colihominis, Butyrivibro proteoclasticus, Akkermansia muciniphila, Lachnospora pectinoschiza, Pedobacter kwangyangensis, Alkaliphilus crotonatoxidans, Lactobacillus salivarius, Anaerivibria lipolyticus, Rhodothermus clarus, Bacteroides stercorirosoris, Ruminocococcus flavefaciens, Bacteroides xylanisolvens, Ruminococcus gnavus, Clostridium termitidis, Clostridium alkalicellulosi, Emticicia oligoraphica, Pseudobutyrivibro xylanivorans, Actinomyces naturae, Peptoniphilus coxii, and Dolichospermum curvum.

6. The gut microbial species as mentioned in claim 5, wherein the composition comprising garcinol, individually or in combination with forskolin increases the viable colony count of Akkermansia muciniphila.

7. The method as in claim 1, wherein said modification of gut microbiota is effective in therapeutic management of diseases selected from the group consisting of obesity, cardiovascular complications, inflammatory bowel disease, crohn's disease, Celiac disease, metabolic syndrome, liver diseases and neurological disorders.

8. The method as in claim 1, wherein the composition is formulated with pharmaceutically/nutraceutically acceptable excipients, adjuvants, diluents or carriers and administered orally in the form of tablets, capsules, syrups, gummies, powders, suspensions, emulsions, chewable, candies and eatables.
